(19) United States
(12) Patent Application Publication
(10) Pub. No.: US 2021/0340252 A1
(43) Pub. Date: Nov. 4, 2021

(54) ANTI-TIM-3 ANTIBODIES AND USES THEREOF

(71) Applicant: WUXI BIOLOGICS IRELAND LIMITED, Dublin (IE)

(72) Inventors: Siwei NIE, Shanghai (CN); Yong ZHENG, Shanghai (CN); Jun PAN, Shanghai (CN); Jianqing XU, Shanghai (CN); Jing LI, Shanghai (CN)

(73) Assignee: WUXI BIOLOGICS IRELAND LIMITED, Dublin (IE)

(21) Appl. No.: 17/312,847

(22) PCT Filed: Dec. 11, 2019
(86) PCT No.: PCT/CN2019/124549

§ 371 Date: Jun. 10, 2021

(30) Foreign Application Priority Data
Dec. 12, 2018 (CN) PCT/CN2018/120631

(51) Int. Cl. C07K 16/28; A61P 35/00

(52) U.S. Cl. CPC C07K 16/2803; C12N 5/12; A61P 35/00

(57) ABSTRACT

Provided in the present disclosure are anti-TIM-3 antibodies, the methods of hybridoma generation, the nucleic acid molecules encoding the anti-TIM-3 antibodies, expression vectors and host cells used for the expression of anti-TIM-3 antibodies. The disclosure further provides the methods for validating the function of antibodies in vitro and the efficacy of antibodies in vivo. The antibodies of the disclosure provide a very potent agent for the treatment of cancers via modulating immune functions.

### RELATED APPLICATIONS

The instant application claims priority to PCT application PCT/CN2018/120631, filed on Dec. 12, 2018, incorporated by reference in its entirety herein.

### SEQUENCE LISTING

The instant application contains a sequence listing which is hereby incorporated by reference in its entirety.

### FIELD OF THE INVENTION

This application generally relates to antibodies. More specifically, the application relates to fully human monoclonal antibodies against TIM-3, a method for preparing the same, and the use of the antibodies.

### BACKGROUND OF THE INVENTION

Increasing evidence from preclinical and clinical results have shown that targeting immune checkpoints is becoming one of the most promising approaches to treat patients with cancers. T cell immunoglobulin mucin-3 (TIM-3), member of the TIM family, is preferentially expressed on activated Th1 cells and cytotoxic CD8 T cells that secrete IFNγ, dendritic cells (DCs), monocytes and NK cells [1]. It is an activation-induced inhibitory molecule and induces the apoptosis of Th1 cells, resulting in T cell exhaustion in chronic viral infection and cancers [2, 3]. It has been suggested that TIM-3 may be a key immune checkpoint in tumor-induced immune suppression [4].

TIM-3 is a type I transmembrane protein that possesses an N-terminal Ig domain of the V type, followed by a mucin domain containing potential sites of glycosylation [5]. Four molecules have been reported as ligands of TIM-3, including carcinoembryonic antigen cell adhesion molecule 1 (CEACAM1), phosphatidylserine (PtdSer), high mobility group protein 1 (HMGB1), and galectine-9 (Gal-9) [6, 7, 8, 9]. Among these ligands, CEACAM1, HMGB1 as well as Gal-9 have been reported to negatively regulate immune response [6, 8, and 10].

CEACAM1, known to be expressed on activated T cells and involved in T cell inhibition, can form cis and trans interaction with TIM-3 to suppress anti-tumor T cell response [6]. HMGB1 binds to DNA released by cells undergoing necrosis and mediates the activation of innate cells through receptor for advanced glycation end (RAGE) products and/or toll-like receptors. By binding to HMGB1, TIM-3 prevents the binding of HMGB1 to DNA, and therefore interferes the function of HMGB1 on activating the innate immune response in tumor tissue [8]. Although the role of Gal-9 on human T cells is controversial, Gal-9 has been shown to bind to mouse TIM-3 and negatively regulate Th-1 immune response. In addition, recently, leukocyte immunoglobulin-like receptor subfamily B member 2 (LILRB2) has been reported to interact with TIM-3 to regulate the function of DCs, macrophages and T cells. The blockage of TIM-3/LILRB2 interaction can enhance the activation of macrophages; increase T cell response and proliferation (US Patent Application No. US20160200815 A1).

It has been suggested that TIM-3 may be a key immune checkpoint in tumor-induced immune suppression, as TIM-3 is expressed on the most suppressed or dysfunctional tumor-infiltrating lymphocytes (TILs) in preclinical models of both solid and hematologic malignancy, as well as patients with advanced melanoma, non-small cell lung cancer (NSCLC) or follicular B-cell non-Hodgkin lymphoma [11, 12]. In multiple preclinical tumor models, the treatment of anti-TIM-3 can dramatically suppress the tumor growth [13].

No therapeutic antibody modulating TIM-3 signaling is commercially available yet. There are some spaces for improvement for antibody against TIM-3 as a therapeutic agent.

In the present disclosure, fully human antibodies against TIM-3 have been generated. The antibodies of the present disclosure bind to human TIM-3 protein with high affinity; have no cross-family reactions to human TIM-1 or TIM-4; block the binding between PtdSer and human TIM-3; and is potent to modulate immune responses in vitro and in vivo.

### SUMMARY OF THE INVENTION

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

The present disclosure provides fully human monoclonal antibodies against TIM-3. It also provides the methods of hybridoma generation using a OmniRat (developed by Open Monoclonal Technology (OMT) Company), the nucleic acid molecules encoding the anti-TIM-3 antibodies, expression vectors and host cells used for the expression of anti-TIM-3 antibodies. The present disclosure further provides the methods for validating the function of antibodies in vitro and in vivo. The antibodies of the present disclosure provide a very potent agent for the treatment of multiple cancers via modulating human immune function.

In some aspects, the present disclosure comprises an isolated antibody, or an antigen-binding portion thereof.

In some embodiments, the isolated antibody or the antigen-binding portion thereof comprises:

A) one or more heavy chain CDRs (HCDRs) selected from the group consisting of:
(i) a HCDR1 comprising SEQ ID NO: 1;
(ii) a HCDR2 comprising one of the amino acid sequences selected from the group consisting of SEQ ID NOs: 2 and 7; and
(iii) a HCDR3 comprising SEQ ID NO: 3;
   B) one or more light chain CDRs (LCDRs) selected from the group consisting of:
(i) a LCDR1 comprising SEQ ID NO: 4;
(ii) a LCDR2 comprising SEQ ID NO: 5; and
(iii) a LCDR3 comprising SEQ ID NO: 6; or
   C) one or more HCDRs of A) and one or more LCDRs of B).

In some embodiments, the isolated antibody or the antigen-binding portion thereof comprises:

A) one or more heavy chain CDRs (HCDRs) selected from the group consisting of:
(i) a HCDR1 as set forth in SEQ ID NO: 1;
(ii) a HCDR2 as set forth in one of the amino acid sequences selected from the group consisting of SEQ ID NOs: 2 and 7; and
(iii) a HCDR3 as set forth in SEQ ID NO: 3;
   B) one or more light chain CDRs (LCDRs) selected from the group consisting of:
(i) a LCDR1 as set forth in SEQ ID NO: 4;
(ii) a LCDR2 as set forth in SEQ ID NO: 5; and
(iii) a LCDR3 as set forth in SEQ ID NO: 6; or
   C) one or more HCDRs of A) and one or more LCDRs of B).

In some embodiments, the isolated antibody or the antigen-binding portion thereof comprises a heavy chain variable region (VH) and a light chain variable region (VL), wherein
(a) the VH comprises:
   (i) a HCDR1 as set forth in SEQ ID NO: 1;
   (ii) a HCDR2 as set forth in SEQ ID NO: 2; and
   (iii) a HCDR3 as set forth in SEQ ID NO: 3; and
(b) the VL comprises:
   (i) a LCDR1 as set forth in SEQ ID NO: 4;
   (ii) a LCDR2 as set forth in SEQ ID NO: 5; and
   (iii) a LCDR3 as set forth in SEQ ID NO: 6.

In some embodiments, the isolated antibody or the antigen-binding portion thereof comprises a heavy chain variable region (VH) and a light chain variable region (VL), wherein
(a) the VH comprises:
   (i) a HCDR1 as set forth in SEQ ID NO: 1;
   (ii) a HCDR2 as set forth in SEQ ID NO: 7; and
   (iii) a HCDR3 as set forth in SEQ ID NO: 3; and
(b) the VL comprises:
   (i) a LCDR1 as set forth in SEQ ID NO: 4;
   (ii) a LCDR2 as set forth in SEQ ID NO: 5; and
   (iii) a LCDR3 as set forth in SEQ ID NO: 6.

In some embodiments, the isolated antibody or the antigen-binding portion thereof comprises: (A) a heavy chain variable region (VH):
(i) comprising the amino acid sequence selected from the group consisting of SEQ ID NOs: 8 and 14;
(ii) comprising an amino acid sequence at least 85%, 90%, or 95% identical to the amino acid sequence selected from the group consisting of SEQ ID NOs: 8 and 14; or
(iii) comprising an amino acid sequence with addition, deletion and/or substitution of one or more amino acids compared with the amino acid sequence selected from the group consisting of SEQ ID NOs: 8 and 14; and/or
(B) a light chain variable region (VL):
(i) comprising the amino acid sequence selected from the group consisting of SEQ ID NOs: 10 and 12;
(ii) comprising an amino acid sequence at least 85%, at least 90%, or at least 95% identical to the amino acid sequence selected from the group consisting of SEQ ID NOs: 10 and 12; or
(iii) comprising an amino acid sequence with addition, deletion and/or substitution of one or more amino acids compared with the amino acid sequence selected from the group consisting of SEQ ID NOs: 10 and 12.

In some embodiments, the isolated antibody or the antigen-binding portion thereof comprises:
(a) a heavy chain variable region comprising the amino acid sequence of SEQ ID NO: 8 and a light chain variable region comprising the amino acid sequence of SEQ ID NO: 10; or
(b) a heavy chain variable region comprising the amino acid sequence of SEQ ID NO: 8 and a light chain variable region comprising the amino acid sequence of SEQ ID NO: 12; or
(c) a heavy chain variable region comprising the amino acid sequence of SEQ ID NO: 14 and a light chain variable region comprising the amino acid sequence of SEQ ID NO: 12.

In some embodiments, the isolated antibody or the antigen-binding portion thereof comprises: a heavy chain variable region as set forth in SEQ ID NO: 8 and a light chain variable region as set forth in SEQ ID NO: 10.

In some embodiments, the isolated antibody or the antigen-binding portion thereof comprises: a heavy chain variable region as set forth in SEQ ID NO: 8 and a light chain variable region as set forth in SEQ ID NO: 12.

In some embodiments, the isolated antibody or the antigen-binding portion thereof comprises: a heavy chain variable region as set forth in SEQ ID NO: 14 and a light chain variable region as set forth in SEQ ID NO: 12.

In some embodiments, an isolated antibody or the antigen-binding portion thereof of the present disclosure competes binding for the same epitope with the isolated antibody or the antigen-binding portion thereof as defined above.

In some embodiments, the isolated antibody or the antigen-binding portion thereof as disclosed herein have one or more of the following properties:
(a) specifically binding to both human TIM-3 protein and cynomolgus monkey TIM-3 protein, e.g. binding to cell surface human TIM-3 with a EC50 of no more than 0.5 nM;
(b) blocking the binding of TIM3 to PtdSer;
(c) enhancing TCR signaling;
(d) inducing production of a cytokine (e.g., IL-2 or IFN-γ) in human CD4+T cells; and
(e) does not mediate ADCC or CDC activity on human TIM-3 expressing cells.

In some embodiments, the isolated antibody or the antigen-binding portion thereof as disclosed herein is a chimeric antibody, a humanized antibody or a fully human antibody. Preferably, the antibody is a fully human monoclonal antibody.

In some aspects, the present disclosure is directed to an isolated nucleic acid molecule, comprising a nucleic acid sequence encoding the heavy chain variable region and/or the light chain variable region of the isolated antibody as disclosed herein.

In some aspects, the present disclosure is directed to a vector comprising the nucleic acid molecule encoding the antibody or antigen-binding portion thereof as disclosed herein.

In some aspects, the present disclosure is directed to a host cell comprising the expression vector as disclosed herein.

In some aspects, the present disclosure is directed to a pharmaceutical composition comprising at least one antibody or antigen-binding portion thereof as disclosed herein and a pharmaceutically acceptable carrier.

In some aspects, the present disclosure is directed to a method for preparing an anti-TIM-3 antibody or antigen-binding portion thereof which comprises expressing the antibody or antigen-binding portion thereof in the host cell and isolating the antibody or antigen-binding portion thereof from the host cell.

In some aspects, the present disclosure is directed to a method of modulating an immune response in a subject, comprising administering the antibody or antigen-binding portion thereof as disclosed herein to the subject such that an immune response in the subject is modulated.

In some aspects, the present disclosure is directed to a method for treating abnormal cell growth in a subject, comprising administering an effective amount of the antibody or antigen-binding portion thereof or the pharmaceutical composition as disclosed herein to the subject.

In some aspects, the present disclosure is directed to a method for inhibiting growth of tumor cells in a subject, comprising administering an effective amount of the antibody or antigen-binding portion thereof or the pharmaceutical composition as disclosed herein to the subject.

In some aspects, the present disclosure is directed to a method for reducing tumor cell metastasis in a subject, comprising administering an effective amount of the antibody or antigen-binding portion thereof or the pharmaceutical composition as disclosed herein to the subject.

In some aspects, the present disclosure is directed to a method for treating or preventing diseases comprising proliferative disorders (such as cancers), immune disorders, inflammatory disease or infectious diseases in a subject comprising administering an effective amount of the antibody or antigen-binding portion thereof or the pharmaceutical composition as disclosed herein to the subject.

In some aspects, the present disclosure is directed to the use of the antibody or antigen-binding portion thereof as disclosed herein in the manufacture of a medicament for treating or preventing diseases comprising proliferative disorders (such as cancers), immune disorders, inflammatory disease or infectious diseases.

In some aspects, the present disclosure is directed to the use of the antibody or antigen-binding portion thereof as disclosed herein in the manufacture of a diagnostic agent for diagnosing diseases comprising proliferative disorders (such as cancers), immune disorders, inflammatory disease or infectious diseases.

In some aspects, the present disclosure is directed to the antibody or antigen-binding portion thereof as disclosed herein for use in treating or preventing diseases comprising proliferative disorders (such as cancers), immune diseases, inflammatory disease or infectious diseases.

In some aspects, the present disclosure is directed to kits or devices and associated methods that employ the antibody or antigen-binding portion thereof as disclosed herein, and pharmaceutical compositions as disclosed herein, which are useful for the treatment of diseases comprising proliferative disorders (such as cancers), immune disorders, inflammatory disease or infectious diseases.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1 is a graph showing the SDS-PAGE analysis of the antibody W3405-2.61.21-uAb-hIgG4K.

FIG. 2 is a graph showing the non-reduced SDS-PAGE analysis of the mutations designed to improve expression.

FIG. 3 is a graph showing the binding of the antibody “W3405-2.61.21-uAb-p1-hIgG4.SPK” to human TIM-3. “Human IgG4K” is an isotype control.

FIG. 4 is a graph showing the binding of the antibody “W3405-2.61.21-uAb-p1-hIgG4.SPK” to CD4⁺ T cells. FIG. 4A shows the binding of the antibody “W3405-2.61.21-uAb-p1-hIgG4.SPK” on activated and non-activated CD4⁺ T cells. FIG. 4B shows the binding curve of the antibody “W3405-2.61.21-uAb-p1-hIgG4.SPK” on activated CD4⁺ T cells.

FIG. 5 is a graph showing the binding specificity of the antibody “W3405-2.61.21-uAb-p1-hIgG4.SPK” to TIM-3. The antibody “W3405-2.61.21-uAb-p1-hIgG4.SPK” binds specifically to human TIM-3 (FIG. 5A), with no cross-reactive binding to human TIM-1 (FIG. 5B) or TIM-4 (FIG. 5C).

FIG. 6 is a graph showing the binding of the antibody “W3405-2.61.21-uAb-p1-hIgG4.SPK” to cynomolgus monkey TIM-3.

FIG. 7 is a graph showing the dose-dependent blockade of PtdSer-TIM-3 interaction by the antibody “W3405-2.61.21-uAb-p1-hIgG4.SPK”.

FIG. 8 is a graph showing the blocking of the effect of TIM-3 on Jurkat cell IL-2 production by the antibody “W3405-2.61.21-uAb-p1-hIgG4.SPK”.

FIG. 9 is a graph showing the effect of the antibody “W3405-2.61.21-uAb-p1-hIgG4.SPK” on IFN-γ production by CD4⁺ T cells.

FIG. 10 is a graph showing the prevention of human CD4⁺ T cell exhaustion induced by THP-1 cells by the antibody “W3405-2.61.21-uAb-p1-hIgG4.SPK”.

FIG. 11 is a graph showing the result of epitope binning. The antibody “W3405-2.61.21-uAb-p1-hIgG4.SPK” competes with WBP340-BMK8 (FIG. 11A), but not BMK6 (FIG. 11B), for binding to human TIM-3.

FIG. 12 is a graph showing the ADCC effect of the antibodies on TIM3 transfectant CHO-K1.

FIG. 13 is a graph showing the CDC effect of antibodies on TIM3 transfectant CHO-K1.

FIG. 14 is a graph showing the stability of the antibody “W3405-2.61.21-uAb-p1-hIgG4.SPK” in human serum.

FIG. 15 is a graph showing the result of efficacy study in NOG mice HCC827 MiXeno™ model.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. Moreover, any section headings used herein are for organizational purposes only and are not to be construed as limiting the subject matter described.

### Definitions

In order to better understand the invention, the definitions and explanations of the relevant terms are provided as follows.

The term “antibody” or “Ab,” as used herein, generally refers to a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (VH) and a heavy chain constant region (C_{H}). A heavy chain constant region consists of 3 domains (C_{H}1, C_{H}² and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each VH and VL consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody speifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen binding fragments of an antibody may be produced by recombinant DNA techniques or by enzymatic or chemical cleavage of an intact antibody. Under some conditions, antigen binding fragments include Fab, Fab′, F(ab′)₂, Fd, Fv, dAb and complementary determining region (CDR) fragments, single chain antibody (e.g. scFv), chimeric antibody, diabody and such polypeptides that comprise at least part of antibody sufficient to confer the specific antigen binding ability on the polypeptides. Antigen binding fragments of an antibody may be obtained from a given antibody (e.g., the monoclonal anti-human TIM-3 antibody provided in the instant application) by conventional techniques known by a person skilled in the art (e.g., recombinant DNA technique or enzymatic or chemical cleavage methods), and may be screened for specificity in the same manner by which intact antibodies are screened.

The term “monoclonal antibody” or “mAb,” as used herein, refer to a preparation of antibody molecules of single molecular composition. A monoclonal antibody displays a single binding specificity and affinity for a particular epitope.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody,” as used herein, refers to an antibody in which the variable region sequences are derived from one species and the constant region sequences are derived from another species, such as an antibody in which the variable region sequences are derived from a mouse antibody and the constant region sequences are derived from a human antibody.

The term “recombinant antibody,” as used herein, refers to an antibody that is prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “anti-TIM-3 antibody” or “TIM-3 antibody” or “antibody against TIM-3,” as used herein, refers to an antibody, as defined herein, capable of binding to a TIM-3 receptor, for example, a human TIM-3 receptor.

The terms “TIM-3,” “TIM-3 receptor,” “TIM-3 protein,” which are used interchangeably herein, is a member of the TIM family, and is preferentially expressed on activated Th1 cells and cytotoxic CD8 T cells that secrete IFNγ, dendritic cells (DCs), monocytes and NK cells. “TIM-3” is a type I transmembrane protein that possesses an N-terminal Ig domain of the V type, followed by a mucin domain containing potential sites of glycosylation.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen, for example, a TIM-3 receptor.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “compete for binding,” as used herein, refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The ability of “inhibit binding,” as used herein, refers to the ability of an antibody or antigen-binding fragment thereof to inhibit the binding of two molecules to any detectable level. In certain embodiments, the binding of the two molecules can be inhibited at least 50% by the antibody or antigen-binding fragment thereof. In certain embodiments, such an inhibitory effect may be greater than 60%, greater than 70%, greater than 80%, or greater than 90%.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are separate from each other in a protein. Antibodies may be screened depending on competitiveness of binding to the same epitope by conventional techniques known by a person skilled in the art. For example, study on competition or cross-competition may be conducted to obtain antibodies that compete or cross-compete with each other for binding to antigens (e.g. RSV fusion protein). High-throughput methods for obtaining antibodies binding to the same epitope, which are based on their cross-competition, are described in an international patent application WO 03/48731.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds an TIM-3 protein is substantially free of antibodies that specifically bind antigens other than TIM-3 proteins). An isolated antibody that specifically binds a human TIM-3 protein may, however, have cross-reactivity to other antigens, such as TIM-3 proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as k phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the invention, human TIM-3 gene was transfected into 293F cells.

The term “hybridoma” and the term “hybridoma cell line,” as used herein, may be used interchangeably. When the term “hybridoma” and the term “hybridoma cell line” are mentioned, they also include subclone and progeny cell of hybridoma.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g. as described in Gazzano-Santoro et al., J. Immunol. Methods 202: 163 (1996), may be performed.

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of TIM-3-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance.

There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Anti-TIM-3 Antibodies

In some aspects, the invention comprises an isolated antibody or an antigen-binding portion thereof.

In the context of the application, the “antibody” may include polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof; and derivatives thereof including Fc fusions and other modifications, and any other immune-reactive molecule so long as it exhibits preferential association or binding with a TIM-3 protein. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). In a preferred embodiment, the antibody is a monoclonal antibody. In a more preferred embodiment, the antibody is a humanized monoclonal antibody or fully human monoclonal antibody.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and art-recognized biochemical and genetic engineering techniques such as described in more detail in An, Zhigiang (ed.) Therapeutic Monoclonal Antibodies: From Bench to Clinic, John Wiley and Sons, 1s ed. 2009; Shire et. al. (eds.) Current Trends in Monoclonal Antibody Development and Manufacturing, Springer Science+Business Media LLC, 1st ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981) each of which is incorporated herein in its entirety by reference. It should be understood that a selected binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also an antibody of this invention. In a preferred embodiment, the anti-human TIM-3 monoclonal antibody is prepared by using hybridoma techniques. Generation of hybridomas is well-known in the art. See, e.g., Harlow and Lane (1988) Antibodies, A Laboratory Manual, Cold Spring Harbor Publications, New York.

Anti-TIM-3 Antibodies with Certain Properties

The antibodies of the present disclosure are characterized by particular functional features or properties of the antibodies. In some embodiments, the isolated antibody or the antigen-binding portion thereof has one or more of the following properties:
(a) specifically binding to both human TIM-3 protein and monkey TIM-3 protein;
(b) blocking the binding of TIM3 to PtdSer;
(c) enhancing TCR signaling; and
(d) inducing production of a cytokine (e.g., IL-2 or IFN-γ) in human CD4+T cells.

The antibody of the disclosure binds to both human and cynomolgus monkey TIM-3 with high affinity. The binding of an antibody of the disclosure to TIM-3 can be assessed using one or more techniques well established in the art, for instance, ELISA. The binding specificity of an antibody of the disclosure can also be determined by monitoring binding of the antibody to cells expressing an TIM-3 protein, e.g., flow cytometry. For example, an antibody can be tested by a flow cytometry assay in which the antibody is reacted with a cell line that expresses human TIM-3, such as CHO cells that have been transfected to express TIM-3 on their cell surface. Other suitable cells for use in flow cytometry assays include anti-CD3-stimulated CD4⁺ activated T cells, which express native TIM-3. Additionally, or alternatively, the binding of the antibody, including the binding kinetics (e.g., Kd value) can be tested in BIAcore binding assays. Still other suitable binding assays include ELISA assays, for example using a recombinant TIM-3 protein. For instance, an antibody of the disclosure binds to a human TIM-3 with a K_{D} of 1×10⁻⁹ M or less, binds to a human TIM-3 with a K_{D} of 5×10⁻¹⁰ M or less, binds to a human TIM-3 with a K_{D} of 2×10⁻¹⁰ M or less, binds to a human TIM-3 protein with a K_{D} of 1×10⁻¹⁰ M or less, binds to a human TIM-3 protein with a K_{D} of 5×10⁻¹¹ M or less, binds to a human TIM-3 protein with a K_{D} of 3×10⁻¹¹ M or less, or binds to a human TIM-3 protein with a K_{D} of 2×10⁻¹¹ M or less.

Further, the antibodies of the present disclosure may block the binding of TIM3 to PtdSer. TIM-3 is known to interact with PtdSer, which tends to be exposed on the surface of apoptotic cells, and can cause immunosuppression. Blockade of a PtdSer-TIM-3 interaction, e.g., using an anti-TIM-3 antibody as described herein may ameliorate or overcome the immunosuppression.

Anti-TIM-3 Antibodies Comprising CDRs

In some embodiments, the isolated antibody or the antigen-binding portion thereof comprises:

A) one or more heavy chain CDRs (HCDRs) selected from the group consisting of:
(i) a HCDR1 comprising SEQ ID NO: 1;
(ii) a HCDR2 comprising one of the amino acid sequences selected from the group consisting of SEQ ID NOs: 2 and 7; and
(iii) a HCDR3 comprising SEQ ID NO: 3;

B) one or more light chain CDRs (LCDRs) selected from the group consisting of:
(i) a LCDR1 comprising SEQ ID NO: 4;
(ii) a LCDR2 comprising SEQ ID NO: 5; and
(iii) a LCDR3 comprising SEQ ID NO: 6; or
   C) one or more HCDRs of A) and one or more LCDRs of B).

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to Kabat numbering system.

In some embodiments, the isolated antibody or the antigen-binding portion thereof comprises:

A) one or more heavy chain CDRs (HCDRs) selected from at least one of the group consisting of:
(i) a HCDR1 as set forth in SEQ ID NO: 1;
(ii) a HCDR2 as set forth in one of the amino acid sequences selected from the group consisting of SEQ ID NOs: 2 and 7; and
(iii) a HCDR3 as set forth in SEQ ID NO: 3;
   B) one or more light chain CDRs (LCDRs) selected from at least one of the group consisting of:
(i) a LCDR1 as set forth in SEQ ID NO: 4;
(ii) a LCDR2 as set forth in SEQ ID NO: 5; and
(iii) a LCDR3 as set forth in SEQ ID NO: 6; or
   C) one or more HCDRs of A) and one or more LCDRs of B).

In a specific embodiment, the isolated antibody or the antigen-binding portion thereof comprises: a heavy chain variable region (VH) and a light chain variable region (VL), and wherein
(a) the VH comprises:
   (i) a HCDR1 as set forth in SEQ ID NO: 1;
   (ii) a HCDR2 as set forth in SEQ ID NO: 2; and
   (iii) a HCDR3 as set forth in SEQ ID NO: 3; and
(b) the VL comprises:
   (i) a LCDR1 as set forth in SEQ ID NO: 4;
   (ii) a LCDR2 as set forth in SEQ ID NO: 5; and
   (iii) a LCDR3 as set forth in SEQ ID NO: 6.

In another specific embodiment, the isolated antibody or the antigen-binding portion thereof comprises a heavy chain variable region (VH) and a light chain variable region (VL), and wherein
(a) the VH comprises:
   (i) a HCDR1 as set forth in SEQ ID NO: 1;
   (ii) a HCDR2 as set forth in SEQ ID NO: 7; and
   (iii) a HCDR3 as set forth in SEQ ID NO: 3; and
(b) the VL comprises:
   (i) a LCDR1 as set forth in SEQ ID NO: 4;
   (ii) a LCDR2 as set forth in SEQ ID NO: 5; and
   (iii) a LCDR3 as set forth in SEQ ID NO: 6.

Anti-TIM-3 Antibodies Comprising a Heavy Chain Variable Region and a Light Chain Variable Region

In some embodiments, the isolated antibody or the antigen-binding portion thereof comprises:

(A) a heavy chain variable region (VH):
(i) comprising the amino acid sequence selected from the group consisting of SEQ ID NOs: 8 and 14;
(ii) comprising an amino acid sequence at least 85%, 90%, or 95% identical to the amino acid sequence selected from the group consisting of SEQ ID NOs: 8 and 14; or
(iii) comprising an amino acid sequence with addition, deletion and/or substitution of one or more amino acids compared with the amino acid sequence selected from the group consisting of SEQ ID NOs: 8 and 14; and/or
   (B) a light chain variable region:
(i) comprising the amino acid sequence selected from the group consisting of SEQ ID NOs: 10 and 12;
(ii) comprising an amino acid sequence at least 85%, at least 90%, or at least 95% identical to the amino acid sequence selected from the group consisting of SEQ ID NOs: 10 and 12; or
(iii) comprising an amino acid sequence with addition, deletion and/or substitution of one or more amino acids compared with the amino acid sequence selected from the group consisting of SEQ ID NOs: 10 and 12.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In a specific embodiment, the isolated antibody or the antigen-binding portion thereof comprises: a heavy chain variable region comprising or consisting of the amino acid sequence of SEQ ID NO: 8 and a light chain variable region comprising or consisting of the amino acid sequence of SEQ ID NO: 10.

In a specific embodiment, the isolated antibody or the antigen-binding portion thereof comprises: a heavy chain variable region comprising or consisting of the amino acid sequence of SEQ ID NO: 14 and a light chain variable region comprising or consisting of the amino acid sequence of SEQ ID NO: 10.

In a specific embodiment, the isolated antibody or the antigen-binding portion thereof comprises: a heavy chain variable region comprising or consisting of the amino acid sequence of SEQ ID NO: 8 and a light chain variable region comprising the amino acid sequence of SEQ ID NO: 12.

In a specific embodiment, the isolated antibody or the antigen-binding portion thereof comprises: a heavy chain variable region comprising or consisting of the amino acid sequence of SEQ ID NO: 14 and a light chain variable region comprising or consisting of the amino acid sequence of SEQ ID NO: 12.

In other embodiments, the amino acid sequences of the heavy chain variable region and/or the light chain variable region can be at least 85%, 86%, 87%, 88%, 89%, 90%, 91%, 92%, 93%, 94%, 95%, 96%, 97%, 98% or 99% identical to the respective sequences set forth above.

In some further embodiments, the isolated antibody or the antigen-binding portion thereof may contain conservative substitution or modification of amino acids in the variable regions of the heavy chain and/or light chain. It is understood in the art that certain conservative sequence modification can be made which do not remove antigen binding. See, e.g., Brummell et al. (1993) Biochem 32:1180-8; de Wildt et al. (1997) Prot. Eng. 10:835-41; Komissarov et al. (1997) J. Biol. Chem. 272:26864-26870; Hall et al. (1992) J. Immunol. 149:1605-12; Kelley and O'Connell (1993) Biochem. 32:6862-35; Adib-Conquy et al. (1998) Int. Immunol. 10:341-6 and Beers et al. (2000) Clin. Can. Res. 6:2835-43.

As described above, the term “conservative substitution,” as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having 0-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

Binning and Epitope Mapping

It will further be appreciated the disclosed antibodies will associate with, or bind to, discrete epitopes or immunogenic determinants presented by the selected target or fragment thereof. In some embodiments, epitope or immunogenic determinants include chemically active surface groupings of molecules such as amino acids, sugar side chains, phosphoryl groups, or sulfonyl groups. In some embodiments, epitopes may have specific three-dimensional structural characteristics, and/or specific charge characteristics. Thus, as used herein the term “epitope” includes any protein determinant capable of specific binding to an immunoglobulin or T-cell receptor or otherwise interacting with a molecule. In some embodiments, an antibody is said to specifically bind (or immune-specifically bind or react) an antigen when it preferentially recognizes its target antigen in a complex mixture of proteins and/or macromolecules. In some embodiments, an antibody is said to specifically bind an antigen when the equilibrium dissociation constant (K_{D}) is less than or equal to 10⁻⁶ M or less than or equal to 10⁻⁷ M, more preferably when the e K_{D} is less than or equal to 10⁻⁸ M, and even more preferably when the K_{D} is less than or equal to 10⁻⁹ M.

Epitopes formed from contiguous amino acids (sometimes referred to as “linear” or “continuous” epitopes) are typically retained upon protein denaturing, whereas epitopes formed by tertiary folding are typically lost upon protein denaturing. In any event an antibody epitope typically includes at least 3, and more usually, at least 5 or 8-10 amino acids in a unique spatial conformation.

In this respect, it will be appreciated that, in some embodiments, an epitope may be associated with, or reside in, one or more regions, domains or motifs of, for example, the TIM-3 protein. Similarly, the art-recognized term “motif” will be used in accordance with its common meaning and shall generally refer to a short, conserved region of a protein that is typically ten to twenty contiguous amino acid residues.

In any event once a desired epitope on an antigen is determined, it is possible to generate antibodies to that epitope, e.g., by immunizing with a peptide comprising the epitope using techniques described in the present disclosure. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that competitively bind with one another, i.e. the antibodies compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

As used herein, the term “binning” refers to methods used to group or classify antibodies based on their antigen binding characteristics and competition. While the techniques are useful for defining and categorizing the antibodies of the present disclosure, the bins do not always directly correlate with epitopes and such initial determinations of epitope binding may be further refined and confirmed by other art-recognized methodology in the art and as described herein. However, it will be appreciated that empirical assignment of the antibodies to individual bins provides information that may be indicative of the therapeutic potential of the disclosed antibodies.

More specifically, one can determine whether a selected reference antibody (or fragment thereof) binds to the same epitope or cross competes for binding with a second test antibody (i.e., is in the same bin) by using methods known in the art and set forth in the Examples herein.

Other compatible epitope mapping techniques include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9: 487-496) (herein specifically incorporated by reference in its entirety).

Nucleic Acid Molecules Encoding Antibodies of the Disclosure

In some aspects, the disclosure is directed to an isolated nucleic acid molecule, comprising a nucleic acid sequence encoding the heavy chain variable region and/or the light chain variable region of the isolated antibody as disclosed herein.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

The isolated nucleic acid encoding the VH region can be converted to a full-length heavy chain gene by operatively linking the VH-encoding nucleic acid to another DNA molecule encoding heavy chain constant regions (CH1, CH2 and CH3). The sequences of human heavy chain constant region genes are known in the art (see e.g., Kabat et al. (1991), supra) and DNA fragments encompassing these regions can be obtained by standard PCR amplification. The heavy chain constant region can be an IgG1, IgG2, IgG3, IgG4, IgA, IgE, IgM or IgD constant region, but more preferably is an IgG1 or IgG4 constant region.

The isolated nucleic acid encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al., supra) and DNA fragments encompassing these regions can be obtained by standard PCR amplification. In preferred embodiments, the light chain constant region can be a kappa or lambda constant region.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In some embodiments, the disclosure is directed to an isolated nucleic acid molecule, comprising a nucleic acid sequence encoding the heavy chain variable region of the isolated antibody as disclosed herein.

In some specific embodiments, the isolated nucleic acid molecule encodes the heavy chain variable region of the isolated antibody and comprises a nucleic acid sequence selected from the group consisting of:

(A) a nucleic acid sequence that encodes a heavy chain variable region as set forth in SEQ ID NO: 8 or 14;

(B) a nucleic acid sequence as set forth in SEQ ID NO: 9 or 15; or

(C) a nucleic acid sequence that hybridized under high stringency conditions to the complementary strand of the nucleic acid sequence of (A) or (B).

In some embodiments, the disclosure is directed to an isolated nucleic acid molecule, comprising a nucleic acid sequence encoding the light chain variable region of the isolated antibody as disclosed herein.

In some specific embodiments, the isolated nucleic acid molecule encodes the light chain variable region of the isolated antibody comprises a nucleic acid sequence selected from the group consisting of:

(A) a nucleic acid sequence that encodes a light chain variable region as set forth in SEQ ID NO: 10 or 12;

(B) a nucleic acid sequence as set forth in SEQ ID NO: 11 or 13; or

(C) a nucleic acid sequence that hybridized under high stringency conditions to the complementary strand of the nucleic acid sequence of (A) or (B).

For example, the nucleic acid molecule is consisted of SEQ ID NO: 9 or 15. Alternatively, the nucleic acid molecule share at least 80% (e.g. at least 85%, 86%, 87%, 88%, 89%, 90%, 91%, 92%, 93%, 94%, 95%, 96%, 97%, 98%, or 99%) sequence identity to SEQ ID NO: 9 or 15. In some specific embodiments, the percentage of identity is derived from the degeneracy of the genetic code, and the encoded protein sequences remain unchanged.

Exemplary high stringency conditions include hybridization at 45° C. in 5×SSPE and 45% formamide, and a final wash at 65° C. in 0.1×SSC. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al, (Eds.), Molecular Cloning: A laboratory Manual. Cold Spring Harbor Laboratory Press: Cold Spring Harbor, N.Y. (1989), pp. 9.47 to 9.51.

Host Cells

Host cells as disclosed in the present disclosure may be any cell which is suitable for expressing the antibodies of the present disclosure, for instance, mammalian cells. Mammalian host cells for expressing the antibodies of the present disclosure include Chinese Hamster Ovary (CHO cells) (including dhfr CHO cells, described in Urlaub and Chasm, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), NSO myeloma cells, COS cells and SP2 cells. In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the antibodies are produced by culturing the host cells for a period of time sufficient to allow for expression of the antibody in the host cells or, secretion of the antibody into the culture medium in which the host cells are grown. Antibodies can be recovered from the culture medium using standard protein purification methods.

Pharmaceutical Compositions

In some aspects, the disclosure is directed to a pharmaceutical composition comprising at least one antibody or antigen-binding portion thereof as disclosed herein and a pharmaceutically acceptable carrier.

Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-TIM-3 antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the antibody or the antigen binding portion thereof of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the present disclosure will comprise multiple doses of the selected drug product over a period of weeks or months. More specifically, the antibody or the antigen-binding portion thereof of the present disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the antibody or the antigen binding portion thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml

### Applications of the Disclosure

The antibodies, antibody compositions and methods of the present disclosure have numerous in vitro and in vivo utilities involving, for example, detection of TIM-3 or enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the T-cell mediated immune response). In a particular embodiment, the methods are particularly suitable for treatment of cancer in vivo. To achieve antigen-specific enhancement of immunity, the anti-TIM-3 antibodies can be administered together with an antigen of interest or the antigen may already be present in the subject to be treated (e.g., a tumor-bearing or virus-bearing subject). When antibodies to TIM-3 are administered together with another agent, the two can be administered in either order or simultaneously.

The present disclosure further provides methods for detecting the presence of human TIM-3 antigen in a sample, or measuring the amount of human TIM-3 antigen, comprising contacting the sample, and a control sample, with a human monoclonal antibody, or an antigen binding portion thereof, which specifically binds to human TIM-3, under conditions that allow for formation of a complex between the antibody or portion thereof and human TIM-3. The formation of a complex is then detected, wherein a difference complex formation between the sample compared to the control sample is indicative of the presence of human TIM-3 antigen in the sample. Moreover, the anti-TIM-3 antibodies of the disclosure can be used to purify human TIM-3 via immunoaffinity purification.

Treatment of Disorders Including Cancers

In some aspects, the present disclosure provides a method of treating a disorder or a disease in a mammal, which comprises administering to the subject (for example, a human) in need of treatment a therapeutically effective amount of the antibody or antigen-binding portion thereof as disclosed herein. The disorder or disease comprises but not limited to, proliferative disorders (such as cancers), immune disorders, inflammatory disease or infectious diseases. For example, the disorder may be a cancer.

A variety of cancers where TIM-3 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer. In another specific embodiment, the cancer is NSCLC.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some other embodiments, the disorder is an autoimmune disease. Examples of autoimmune diseases that may be treated with the antibody or antigen-binding portion thereof include autoimmune encephalomyelitis, lupus erythematosus, and rheumatoid arthritis. The antibody or the antigen-binding portion thereof may also be used to treat or prevent infectious disease, inflammatory disease (such as allergic asthma) and chronic graft-versus-host disease.

Stimulation of an Immune Response

In some aspects, the disclosure also provides a method of enhancing (for example, stimulating) an immune response in a subject comprising administering an antibody or an antigen binding portion thereof of the disclosure to the subject such that an immune response in the subject is enhanced. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4⁺ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-2 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the antibody or an antigen binding portion thereof is used to enhance the immune response of a human to a microbial pathogen (such as a virus). In another embodiment, the antibody or an antigen binding portion thereof is used to enhance the immune response of a human to a vaccine. In one embodiment, the method enhances a cellular immune response, particularly a cytotoxic T cell response. In another embodiment, the cellular immune response is a T helper cell response. In still another embodiment, the immune response is a cytokine production, particularly IFN-γ production or IL-2 production. The antibody or an antigen binding portion thereof may be used to enhance the immune response of a human to a microbial pathogen (such as a virus) or to a vaccine.

The antibody or the antigen-binding portion thereof may be used alone as a monotherapy, or may be used in combination with chemical therapies or radiotherapies.

Combined Use with Chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib,sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Diagnosis

The disclosure provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with an antibody as described herein and detecting presence or absence, or level of association, of the antibody to bound or free target molecules in the sample. In some embodiments, the antibody will comprise a detectable label or reporter molecule as described herein.

In some embodiments, the association of the antibody with particular cells in the sample can denote that the sample may contain tumorigenic cells, thereby indicating that the individual having cancer may be effectively treated with an antibody as described herein.

Samples can be analyzed by numerous assays, for example, radioimmunoassays, enzyme immunoassays (e.g. ELISA), competitive-binding assays, fluorescent immunoassays, immunoblot assays, Western Blot analysis and flow cytometry assays. Compatible in vivo theragnostic or diagnostic assays can comprise art recognized imaging or monitoring techniques, for example, magnetic resonance imaging, computerized tomography (e.g. CAT scan), positron tomography (e.g., PET scan), radiography, ultrasound, etc., as would be known by those skilled in the art.

Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

Sequence Listing Summary

Appended to the instant application is a sequence listing comprising a number of nucleic acid and amino acid sequences. The following Table A, B and C provides a summary of the included sequences.

Three illustrative antibodies as disclosed herein, which are anti-TIM-3 monoclonal antibodies, are designated as “W3405-2.61.21”, “W3405-2.61.21 (V87E)” (also referred to as “W3405-2.61.21-uAb-hIgG4.SPK (V87E)” or “W3405”) and “W3405-2.61.21-uAb-p1” (also referred to as “W3405-2.61.21-uAb-p1-hIgG4.SPK”), respectively. “W3405-2.61.21” serves as the parental anti-TIM-3 antibody, “W3405-2.61.21 (V87E)” is the expression optimized antibody on the basis of the parental antibody, and “W3405-2.61.21-uAb-p1” is the final PTM (“post translational modification”) removed lead antibody.

TABLE A
CDR amino acid sequences

| | | CDR1 | CDR2 | CDR3 |
|---|---|---|---|---|
| W3405- | VH | SEQ ID | SEQ ID | SEQ ID |
| 2.61.21 | | NO: 1 | NO: 2 | NO: 3 |
| | | GFTFSNYAMS | SISDNGG | DFGDSPGY |
| | | | TAYHADS | |
| | | | VQG | |
| | VL | SEQ ID | SEQ ID | SEQ ID |
| | | NO: 4 | NO: 5 | NO: 6 |
| | | KSSQSVLYSF | WASTRES | QQYYSSPLT |
| | | KNKNYLA | | |
| W3405- | VH | SEQ ID | SEQ ID | SEQ ID |
| 2.61.21 | | NO: 1 | NO: 2 | NO: 3 |
| (V87E) | | GFTFSNYAMS | SISDNGG | DFGDSPGY |
| | | | TAYHADS | |
| | | | VQG | |
| | VL | SEQ ID | SEQ ID | SEQ ID |
| | | NO: 4 | NO: 5 | NO: 6 |
| | | KSSQSVLYSF | WASTRES | QQYYSSPLT |
| | | KNKNYLA | | |
| W3405- | VH | SEQ ID | SEQ ID | SEQ ID |
| 2.61.21- | | NO: 1 | NO: 7 | NO: 3 |
| uAb-p1 | | GFTFSNYAMS | SISDQGG | DFGDSPGY |
| | | | TAYHADS | |
| | | | VQG | |
| | VK | SEQ ID | SEQ ID | SEQ ID |
| | | NO: 4 | NO: 5 | NO: 6 |
| | | KSSQSVLYSF | WASTRES | QQYYSSPLT |
| | | KNKNYLA | | |

TABLE B
Variable region amino acid sequences

| | VH | VL |
|---|---|---|
| W3405- | SEQ ID NO: 8 | SEQ ID NO: 10 |
| 2.61.21 | EVQLLEAGGGPVQPGGSLR | DIVMTQSPDSLAVSLGERATIN |
| | LSCAAAGFTFSNYAMSWVR | CKSSQSVLYSFKNKNYLAWYQQ |
| | QAPGKGLEWVSSISDNGGT | KPGQPPKLLIYWASTRESGVPD |
| | AYHADSVQGRFTISRDNSK | RFSGSGSGTDFTLTISSLQAVD |
| | STLYLQMNSLRAEDTAVYY | VAVYYCQQYYSSPLTFGGGTKV |
| | CAKDFGDSPGYWGQGTLVT | EIK |
| | VSS | |
| W3405- | SEQ ID NO: 8 | SEQ ID NO: 12 |
| 2.61.21 | EVQLLEAGGGPVQPGGSLR | DIVMTQSPDSLAVSLGERATIN |
| (V87E) | LSCAAAGFTFSNYAMSWVR | CKSSQSVLYSFKNKNYLAWYQQ |
| | QAPGKGLEWVSSISDNGGT | KPGQPPKLLIYWASTRESGVPD |
| | AYHADSVQGRFTISRDNSK | RFSGSGSGTDFTLTISSLQAED |
| | STLYLQMNSLRAEDTAVYY | VAVYYCQQYYSSPLTFGGGTKV |
| | CAKDFGDSPGYWGQGTLVT | EIK |
| | VSS | |
| W3405- | SEQ ID NO: 14 | SEQ ID NO: 12 |
| 2.61.21- | EVQLLEAGGGPVQPGGSLR | DIVMTQSPDSLAVSLGERATIN |
| uAb-p1 | LSCAAAGFTFSNYAMSWVR | CKSSQSVLYSFKNKNYLAWYQQ |
| | QAPGKGLEWVSSISDQGGT | KPGQPPKLLIYWASTRESGVPD |
| | AYHADSVQGRFTISRDNSK | RFSGSGSGTDFTLTISSLQAED |
| | STLYLQMNSLRAEDTAVYY | VAVYYCQQYYSSPLTFGGGTKV |
| | CAKDFGDSPGYWGQGTLVT | EIK |
| | VSS | |

TABLE C
Variable region nucleotide sequences

| | VHnu (heavy chain | VLnu (light chain |
|---|---|---|
| | variable region | variable region |
| | nucleotide | nucleotide |
| | sequences) | sequences) |
| W3405- | SEQ ID NO: 9 | SEQ ID NO: 11 |
| 2.61.21 | GAGGTGCAGTTGTTGGAGG | GACATCGTGATGACCCAGTC |
| | CTGGGGGAGGCCCGGTACA | TCCAGACTCCCTGGCTGTGT |
| | GCCTGGGGGGTCCCTGAGA | CTCTGGGCGAGAGGGCCACC |
| | CTCTCCTGTGCAGCCGCTG | ATCAACTGCAAGTCCAGCCA |
| | GATTCACCTTTAGCAACTA | GAGTGTTTTATACAGCTTCA |
| | TGCCATGAGCTGGGTCCGG | AGAATAAGAACTACTTAGCT |
| | CAGGCTCCAGGGAAGGGGC | TGGTACCAGCAGAAACCAGG |
| | TGGAATGGGTCTCAAGTAT | ACAGCCTCCTAAGCTGCTCA |
| | TAGTGACAATGGTGGGACC | TTTACTGGGCATCTACCCGG |
| | GCATACCACGCAGACTCCG | GAATCCGGGGTCCCTGACCG |
| | TGCAGGGCCGATTCACCAT | ATTCAGTGGCAGCGGGTCTG |
| | CTCCAGAGACAATTCCAAG | GGACAGATTTCACTCTCACC |
| | AGCACGCTGTATCTACAAA | ATCAGCAGCCTGCAGGCTGT |
| | TGAACAGCCTGAGAGCCGA | AGATGTGGCAGTTTATTACT |
| | GGACACGGCCGTATATTAC | GTCAGCAATATTATAGTTCT |
| | TGTGCGAAAGACTTCGGTG | CCGCTCACTTTCGGCGGAGG |
| | ACTCCCCGGGCTACTGGGG | GACCAAGGTGGAGATCAAA |
| | CCAGGGAACCCTGGTCACC | |
| | GTCTCCTCA | |
| W3405- | SEQ ID NO: 9 | SEQ ID NO: 13 |
| 2.61.21 | GAGGTGCAGTTGTTGGAGG | GACATCGTGATGACCCAGTC |
| (V87E) | CTGGGGGAGGCCCGGTACA | TCCAGACTCCCTGGCTGTGT |
| | GCCTGGGGGGTCCCTGAGA | CTCTGGGCGAGAGGGCCACC |
| | CTCTCCTGTGCAGCCGCTG | ATCAACTGCAAGTCCAGCCA |
| | GATTCACCTTTAGCAACTA | GAGTGTTTTATACAGCTTCA |
| | TGCCATGAGCTGGGTCCGG | AGAATAAGAACTACTTAGCT |
| | CAGGCTCCAGGGAAGGGGC | TGGTACCAGCAGAAACCAGG |
| | TGGAATGGGTCTCAAGTAT | ACAGCCTCCTAAGCTGCTCA |
| | TAGTGACAATGGTGGGACC | TTTACTGGGCATCTACCCGG |
| | GCATACCACGCAGACTCCG | GAATCCGGGGTCCCTGACCG |
| | TGCAGGGCCGATTCACCAT | ATTCAGTGGCAGCGGGTCTG |
| | CTCCAGAGACAATTCCAAG | GGACAGATTTCACTCTCACC |
| | AGCACGCTGTATCTACAAA | ATCAGCAGCCTGCAGGCTGA |
| | TGAACAGCCTGAGAGCCGA | AGATGTGGCAGTTTATTACT |
| | GGACACGGCCGTATATTAC | GTCAGCAATATTATAGTTCT |
| | TGTGCGAAAGACTTCGGTG | CCGCTCACTTTCGGCGGAGG |
| | ACTCCCCGGGCTACTGGGG | GACCAAGGTGGAGATCAAA |
| | CCAGGGAACCCTGGTCACC | |
| | GTCTCCTCA | |
| W3405- | SEQ ID NO: 15 | SEQ ID NO: 13 |
| 2.61.21- | GAGGTGCAGTTGTTGGAGG | GACATCGTGATGACCCAGTC |
| uAb-p1 | CTGGGGGAGGCCCGGTACA | TCCAGACTCCCTGGCTGTGT |
| | GCCTGGGGGGTCCCTGAGA | CTCTGGGCGAGAGGGCCACC |
| | CTCTCCTGTGCAGCCGCTG | ATCAACTGCAAGTCCAGCCA |
| | GATTCACCTTTAGCAACTA | GAGTGTTTTATACAGCTTCA |
| | TGCCATGAGCTGGGTCCGG | AGAATAAGAACTACTTAGCT |
| | CAGGCTCCAGGGAAGGGGC | TGGTACCAGCAGAAACCAGG |
| | TGGAATGGGTCTCAAGTAT | ACAGCCTCCTAAGCTGCTCA |
| | TAGTGACCAGGGTGGGACC | TTTACTGGGCATCTACCCGG |
| | GCATACCACGCAGACTCCG | GAATCCGGGGTCCCTGACCG |
| | TGCAGGGCCGATTCACCAT | ATTCAGTGGCAGCGGGTCTG |
| | CTCCAGAGACAATTCCAAG | GGACAGATTTCACTCTCACC |
| | AGCACGCTGTATCTACAAA | ATCAGCAGCCTGCAGGCTGA |
| | TGAACAGCCTGAGAGCCGA | AGATGTGGCAGTTTATTACT |
| | GGACACGGCCGTATATTAC | GTCAGCAATATTATAGTTCT |
| | TGTGCGAAAGACTTCGGTG | CCGCTCACTTTCGGCGGAGG |
| | ACTCCCCGGGCTACTGGGG | GACCAAGGTGGAGATCAAA |
| | CCAGGGAACCCTGGTCACC | |
| | GTCTCCTCA | |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Benchmark Antibodies and Cell Lines

### 1.1 Preparation of Materials

Information on the commercially available materials used in the examples are provided in Table 1.

TABLE 1

| | | Catalog Number |
|---|---|---|
| Materials | Vendor | (Cat.) |
| F12-K nutrient mixture (1×) | Life Technologies | 21127-022 |
| FreeStyle 293 Expression | Gibco | 12338026 |
| Medium | | |
| Expi293 expression system kit | ThermoFisher | A14527 |
| Expi293F cells | Thermo Fisher | A14528 |
| Lipofectamine 2000 | invitrogen | 11668019 |
| FBS | Hyclone | RBC 35932 |
| Blasticidin S HCl | Life Technologies | 1612810 |
| Anti-PE Microbeads | Miltenyi | 013-048-801 |
| Ni-NTA column | GE | 175248 |
| Protein A column | GE | 175438 |
| Protein G column | GE | 170618 |
| PlasFect | Bioline | BIO-46026 |
| Size exclusion column | GE Healthcare | 17104301 |
| RNeasy Plus Mini Kit | QIAGEN | 74134 |
| SuperScript III First-Strand | Invitrogen | 18080400 |
| Synthesis SuperMix | | |
| Premix Ex Taq hot start | TaKaRa | RR030A |
| DNA Gel Extraction Kit | Axygen | AP-GX-250 |
| pMD 18-T vector | TaKaRa | 6011 |
| Biacore 8K | GE | NA |
| Series S Sensor Chip CM5 | GE | 29-1496-03 |
| Amine Coupling Kit | GE | BR100050 |
| 10 × HBS-EP+ | GE | BR100669 |
| anti-human Fc IgG | Jackson | 109-005-098 |
| ProteOn XPR36 | Bio-Rad | NA |
| GLM chip | Bio-Rad | 176-5012 |
| ProteOn Amine Coupling Kit | Bio-Rad | 176-2410 |
| HRP goat anti-rat IgG Fc | Bethyl | A110-236P |
| Alexa647 goat anti-rat IgG Fc | Jackson Immuno | 112-606-071 |
| | Research | |
| PE mouse anti human CD366 | eBioscience | 12-3109-41 |
| R-PE goat anti-human IgG Fc | Jackson Immuno | 109-115-098 |
| | Research | |
| PE goat anti-mouse IgG Fc | Abcam | Ab98742 |
| PE goat anti-mouse IgG Fc | Bethyl | A90-239PE |
| HRP goat anti-human IgG Fc | Bethyl | A80-304P |
| HRP goat anti-Human IgG, | Jackson Immuno | 109-035-097 |
| F(ab′)₂ | Research | |
| Streptavidin-HRP | Invitrogen | SNN1004 |
| HRP mouse anti-His antibody | GenScript | A00612 |
| Human TIM-3, His tag | Sino Biological | 10390-H08H |
| Human TIM-1, His tag | Sino Biological | 11051-H07H |
| Human TIM-4, His tag | Sino Biological | 12161-H08H |
| Cynomolgus TIM-3, hFc tag | Sino Biological | 90312-C02H |
| Anti Human TIM1 Mab | Sino Biological | 11051-MM04 |
| Anti Human TIM4 Rabbit Mab | Sino Biological | 12161-R101 |
| Ficoll | Stem Cell | 07861 |
| Recombinant human GM-CSF | Amoytop Biotech | S10980039 |
| Recombinant human IL-4 | R&D | 204-IL-010 |
| Human CD4+ T Cell | Stem Cell | 19052 |
| Enrichment kit | | |
| Human CD14 Microbeads | Miltenyi Biotec | 130-050-201 |
| Human CD56 MicroBeads | Miltenyi Biotec | 130-050-401 |
| SKBR3 | ATCC | HTB-30 |
| Raji | ATCC | CCL-86 |
| Jurkat E6-1 | ATCC | TIB-152 |
| Cytotoxicity Detection | Roche | 04744934001 |
| Kit (LDH) | | |
| Human Serum Complement | Quidel | A112 |
| CellTiter Glo Kit | Promega | G7573 |
| Recombinant human IFN-γ | PeproTech | 300-02 |
| Human IFN-γ capture | Pierce | M700A |
| antibody | | |
| Human IFN-γ detection | Pierce | M701B |
| antibody | | |
| THP-1 | ATCC | TIB-202 |
| PE Mouse Anti-Human IL-2 | BD biosciences | 340450 |
| IFN-γ (human) AlphaLISA | Perkin-Elmer | AL217F |
| Detection Kit | | |
| SE Cell Line 4D-Nucleofector ® | Lonza | V4XC-1012 |
| X Kit | | |
| hTIM-3 KI C57BL/6 | Cavensbiogle | NA |
| MC38 | NTCC | NA |
| NOG | Beijing Vital | 408 |
| | River | |
| HCC827 | ATCC | CRL-2868 |

### 1.2 Production of Antigens

DNA sequences encoding truncated (ECD and transmembrane) or full length of human TIM-3 (GenBank Accession No. NM_032782.3), mouse TIM-3 (GenBank Accession No. NM_134250.2) and cynomolgus monkey TIM-3 (GenBank Accession No. EHH54703.1) were synthesized in Sangon Biotech (Shanghai, China), and then subcloned into modified pcDNA3.3 expression vectors with different tag (such as 6×his, AVI-6×his, human Fc, or mouse Fc) in C-terminal. The expression vectors were purified for use.

Expi293 cells were transfected with the purified expression vectors. Cells were cultured for 5 days and supernatant was collected for protein purification using Ni-NTA column, Protein A column or Protein G column. The obtained human TIM-3.ECD.MBPAVIHIS and mouse TIM-3.ECD.mFc were analyzed by SDS-PAGE and SEC, and then stored at −80° C.

### 1.3 Production of Benchmark Antibodies

Two benchmark antibodies were generated and applied as positive controls in the examples. One benchmark antibody is the antibody named as “ABTIM3-hum11” in U.S. Pat. No. 9,605,070 B2, which is referred to as “WBP340-BMK8” or “W340.BMK8” or “W340.BMK8.uIgG4” in the present disclosure. The second benchmark antibody is the antibody named as “mAb15” in US Patent Application No. US20160200815 A1, which is referred to as “WBP340-BMK6” or “WBP340-BMK6.IgG4” in the present disclosure. DNA sequences encoding the variable regions of ABTIM3-hum11 (WBP340-BMK8) and mAb15 (WBP340-BMK6) were synthesized in Sangon Biotech (Shanghai, China), and then subcloned into modified plasmids pcDNA3.3 expression vectors with the constant region of human IgG4 (S228P).

The plasmids containing VH and VL genes were co-transfected into Expi293 cells. Cells were cultured for 5 days and supernatant was collected for protein purification using Protein A column or Protein G column. The obtained antibodies were analyzed by SDS-PAGE and SEC, and then stored at −80° C.

### 1.4 Cell Pool/Line Generation

Using Lipofectamine 2000, CHO-K1 or 293F cells were transfected with the expression vector containing gene encoding full length human TIM-3, mouse TIM-3 or cynomolgus monkey TIM-3. Cells were cultured in medium containing proper selection marker. Human TIM-3 high expression stable cell line (referred to as “W340-CHO-K1.hProl.G2” herein), lower expression stable cell line (referred to as “W340-CHO-K1.hProl.H1” herein) and mouse TIM-3 high expression stable cell line (referred to as “WBP340.CHO-K1.mProl.D3” herein), cynomolgus monkey TIM-3 high expression stable cell line (referred to as “W340-293F.cynoProl.FL-17” herein), lower expression stable cell line (referred to as “W340-293F.cynoProl.FL-4” herein) were selected after limited dilution.

Jurkat E6-1 cells were transfected with plasmid IL-2P Luc by SE Cell Line 4D-Nucleofector® X Kit according to the manufacturer's protocol. 48 hours after transfection, Hygromycin was added to the cell culture to select Jurkat E6-1 cells stably transfected with IL-2P Luc (referred to as “Jurkat E6-1.IL-2P cells” herein). The plasmid containing full length human TIM-3 (“hTIM-3”) was then transfected to Jurkat E6-1.IL-2P cells using the same method. 48 hours after transfection, Blasticidin S was added to the cell culture to develop the stable cell pool of Jurkat E6-1.IL-2P.hTIM-3. Stable cell lines were obtained by limited dilution.

### Example 2

### Antibody Hybridoma Generation

### 2.1 Immunization

OMT rats (transgenic rats having recombinant immunoglobulin loci, as described and produced in U.S. Pat. No. 8,907,157 B2), 10˜11 weeks of age, were immunized weekly by footpad and subcutaneous injections with 25 μg/animal of hTIM-3.ECD.mFc or 25 μg/animal of mTIM-3.ECD.hFc in adjuvant alternately.

### 2.2 Serum Titer Detection

Post the 4^{th} immunization, serum samples from the immunized OMT rats were collected and examined every two weeks. Anti-hTIM-3 and anti-mTIM-3 antibody titers in the serum samples were determined by ELISA. Briefly, the plates coated with hTIM-3.ECD.His or mTIM-3.ECD.His were co-incubated with diluted rat serum (first 1:100 by volume, then 3-fold dilution in 2% BSA/PBS) for two hours. Goat anti rat-IgG-Fc-HRP was used as secondary antibody. The color was developed by dispensing 100 μL of TMB substrate, and then stopped by 100 μL of 2N HCl. The absorbance was read at 450 nM using a microplate spectrophotometer.

The serum titers of the immunized OMT rats for human TIM-3 and mouse TIM-3 are shown in Table 2 and Table 3, respectively.

TABLE 2
OMT rat serum titer for human TIM-3

| | Human TIM-3 titer by ELISA | | | | | | | |
|---|---|---|---|---|---|---|---|---|
| Animal | | 1^{st} | 2^{nd} | 3^{rd} | 4^{th} | 5^{th} | 6^{th} | 7^{th} |
| ID # | Pre-bleed | bleed | bleed | bleed | bleed | bleed | bleed | bleed |
| 1 | <100 | 24300 | 72900 | 24300 | 24300 | 218700 | 218700 | ~218700 |
| 2 | <100 | 72900 | ~218700 | ~218700 | ~218700 | NA | NA | NA |
| 3 | <100 | ~24300 | ~72900 | 24300 | 24300 | NA | NA | NA |

TABLE 3
OMT rat serum titer for mouse TIM-3

| | Mouse TIM-3 titer by ELISA | | | | | | | |
|---|---|---|---|---|---|---|---|---|
| Animal | | 1^{st} | 2^{nd} | 3^{rd} | 4^{th} | 5^{th} | 6^{th} | 7^{th} |
| ID # | Pre-bleed | bleed | bleed | bleed | bleed | bleed | bleed | bleed |
| 1 | <100 | ~300 | ~2700 | ~2700 | 2700 | 900 | 24300 | 24300 |
| 2 | <100 | 2700 | 72900 | ~72900 | ~72900 | NA | NA | NA |
| 3 | <100 | ~900 | 8100 | 8100 | 24300 | NA | NA | NA |

Rat #1 was euthanized after the 7^{th} bleed and the lymph nodes were collected for fusion.

### 2.3 Hybridoma Generation

When the serum antibody titer was sufficiently high, the OMT rats were given a final boost with both human and mouse TIM-3 ECD proteins in D-PBS without adjuvant. On the day of fusion, lymph nodes were removed from the immunized OMT rats under sterile condition, and prepared into single cell suspension. The isolated cells were then mixed with myeloma cell SP2/0 at a ratio of 1:1. Electro cell fusion was performed using BTX 2001 Electro cell manipulator according to manufacturer's instruction. The cells were then seeded in 96-well plates at the density of 1×10⁴ cells/well, and cultured at 37° C., 5% CO₂, until ready for screening.

### 2.4 Antibody Screening

Human TIM-3 binding ELISA was used as the first screen method to test the binding of hybridoma supernatants to human TIM-3 protein. Briefly, hybridoma supernatant samples, positive control and negative control were added into plates pre-coated with hTIM-3.ECD.His, and cultured for 2 hours. Goat anti rat-IgG-Fc-HRP was used as secondary antibody to detect the binding of rat antibodies onto the plates. The color was developed by dispensing 50 μL of TMB substrate, and then stopped by 50 μL of 2N HCl. The absorbance was read at 450 nM using a microplate spectrophotometer. Samples that had A450≥0.2 were considered positive hTIM-3 binders (NC: 0.05˜0.06).

In order to confirm the initial binding results, the positive hybridoma line was further tested by FACS using WBP340.CHO-K1.hProl.G2 as follow: hybridoma supernatants were added to the cells, and the binding of rat antibodies onto the surface of the cells were detected by Alexa647-labeled goat anti-rat antibody. The MFI was evaluated by a flow cytometer and analyzed by FlowJo. Antibody binding to parental CHO-K1 cells was used as negative control.

Through primary and secondary binding screening, 10 positive cell lines were selected for subcloning.

### 2.5 Hybridoma Subcloning

Once specific binding was verified through first and confirmation screening, the positive hybridoma cells were subcloned to get monoclonal anti-hTIM-3 antibodies by using semi-solid medium approach. The positive clones were confirmed by binding ELISA and FACS against human TIM-3 as described above. The exhausted supernatant of selected single clones was collected for hybridoma antibody purification.

### 2.6 Hybridoma Sequencing

Total RNA was isolated from hybridoma cells by using RNeasy Plus Mini Kit and first strand cDNA was prepared as follows:

cDNA amplification reaction (20 μL)

| Component | Amount | |
|---|---|---|
| Up to 5 μg total RNA | 5 | μL |
| Primer (50 μM oligo(dT)₂₀/50 ng/μL random hexamers) | 1 μL/1 μL | |
| Annealing Buffer | 1 | μL |

Bring the volume to 8 μL using RNase/DNase-free water
65° C. for 5 min, then immediately place on ice for at least 1 minute

| | | |
|---|---|---|
| 2X First-Strand Reaction Mix | 10 | μL |
| SuperScript ™ III/RNaseOUT ™ Enzyme Mix | 2 | μL |

cDNA amplification reaction condition

| | Step 1 | Step 2 | Step 3 | Step 4 |
|---|---|---|---|---|
| Temperature (° C.) | 25 | 50 | 85 | 4 |
| Time | 10 min | 50 min | 5 min | ∞ |

Antibody VH and VL genes were amplified from cDNA using 3′-constant region degenerated primer and 5′-degenerated primer sets, which are complementary to the upstream signal sequence-coding region of Ig variable sequences. The PCR reaction was done as follows:

PCR reaction system (50 μL)

| Component | Amount | |
|---|---|---|
| cDNA | 2.0 | μL |
| Premix Ex Taq | 25 | μL |
| 5′- degenerated primer sets (10 pM) | 2.5 | μL |
| 3′- constant region degenerated primer (10 pM) | 1 | μL |
| ddH₂O | 19.5 | μL |

PCR reaction condition

| | Step 1 | Step 2 | Step 3 | Step 4 | Step 5 |
|---|---|---|---|---|---|
| Temperature | 95 | 94 | 58 | 72 | 72 |
| (° C.) | | | | | |
| Time | 4 min | 45 sec | 45 sec | 1 min | 10 min |

| | | | |
|---|---|---|---|
| Cycles | NA | 30 | NA |

PCR product (10 μL) was ligated into pMD18-T vector and 10 μL of the ligation product was transformed into Top10 competent cells. Transformed cells were plated on 2-YT+Cab plates and incubated overnight at 37° C. 15 positive clones were randomly picked for sequencing by Shanghai Biosune Biotech Co., Ltd.

Through a serial of screening assays, one hybridoma lead antibody, “W3405-2.61.21,” was selected and served as the parental antibody for the following optimization.

### Example 3

### Antibody Optimization

### 3.1 Fully Human Antibody Construction

W3405-2.61.21 VH and VL genes were re-amplified with cloning primers containing appropriate restriction sites. DNA sequence encoding light chain variable region of WBP3405-2.61.21 with the human IgG4 light chain on the C-terminal was cloned into a modified pcDNA3.3 expression vector. DNA sequence encoding heavy chain variable region of WBP3405-2.61.21 with the constant region of human IgG4 (S228P) heavy chain on the C-terminal was cloned into a modified pcDNA3.3 expression vector, to express a fully human antibody named “W3405-2.61.21-uAb-hIgG4K” or “W3405-2.61.21-uAb-hIgG4.SPK” herein.

### 3.2 Optimization to Improve Expression Level

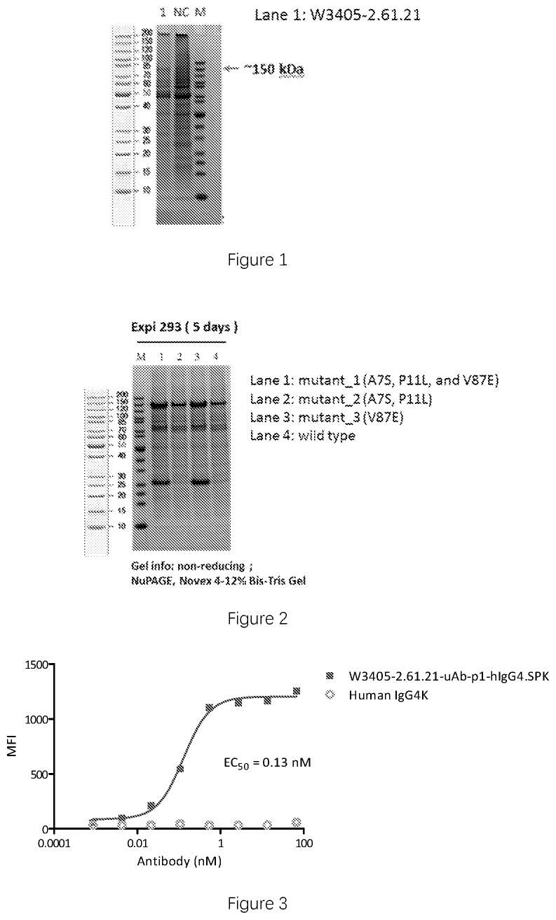

The antibody W3405-2.61.21-uAb-hIgG4K exhibited a markedly low expression level when transiently expressed in Expi293 cells. FIG. 1 showed the SDS-PAGE results of the supernatant of W3405-2.61.21-uAb-hIgG4K transiently expressed in 350 mL Expi293 cells, where only a very light band of the correct molecular weight was observed. The yield of the antibody after protein A purification was only 12 mg/L, which is far less than that of a regular monoclonal antibody produced in Expi293 transient expression (>100 mg/L).

In order to improve the expression level of the antibody W3405-2.61.21-uAb-hIgG4.SPK, the amino acid sequences of the VH and VL of W3405-2.61.21 were analyzed. A statistical analysis on the propensities of all 20 amino acid types at each residue position was conducted by using the antibody database curated by Discovery Studio software. Positions with very rare amino acid types were identified. Two were in the heavy chain variable region: A7 (Kabat: 7) and P11 (Kabat: 11). One was in the light chain variable region: V87 (Kabat: 81). The unusual amino acids were mutated to high propensity types by mutagenesis primers. Val 87 (kabat: 81) was replaced by Glu in light chain and Ala 7 (kabat: 7) and Pro 11 (kabat: 11) are replaced by Ser and Leu in heavy chain, respectively.

Three variants, mutant_1, mutant_2 and mutant_3 were designed. Mutant_1 replaced all 3 residues by their corresponding common amino acid types (A7S, P11L, and V87E), mutant 2 replaced two residues in the heavy chain (A7S and P11L), and mutant 3 just replaced one residue in the light chain (V87E). Variable gene of W3405-2.61.21-uAb-hIgG4.SPK was used as template. The mutation was verified by sequencing. The mutation plasmids, codon optimized plasmids and parental plasmids were co-transfected into Expi293 cells using Expi293 expression system kit, according to the manufacturer's instructions. Five days after transfection, the supernatants were collected and analysis by non-reducing SDS-PAGE. Large-scale transfections up to 100-300 mL were scaled linearly.

Specifically, the three mutants as well as the wild type antibody W3405-2.61.21-uAb-hIgG4.SPK were all transiently expressed in Expi293 cells at 5 mL scale to make a side-by-side comparison. As shown in the supernatant SDS-PAGE in FIG. 2, mutant_1 and mutant_3 exhibited obvious enhancement in expression titer, while the mutant_2-modified heavy chain showed no effects. These results verified that light chain V87 (Kabat: 81) was the only critical residue that prevented the antibody from being properly produced. This discovery was further confirmed by the data from a larger scale production experiment (transient transfection 120 mL Expi293 cells), where the yield of mutant_3 achieved 252.5 mg/L after Protein A purification. This was about 21-fold increase compared to that of the wild type antibody produced earlier.

### 3.3 PTM Removal

A potential PTM site “NG” was identified in the VH-CDR2 region. PTM site removing mutations were introduced by site-directed mutagenesis using QuickChange mutagenesis kit (Agilent Genomics) according to the manufacturer's protocol. Antisense mutagenic nucleotides were designed to introduce the following mutations: N→Q, G→A, the variable gene of W3405-2.61.21-uAb-hIgG4.SPK (V87E) was used as template. Mutations were verified by sequencing. The PTM removed variants were expressed, purified; and the binding affinity to human TIM-3 was examined by SPR.

The p1 variant (N→Q) showed comparable affinity to human TIM-3 as W3405-2.61.21-uAb-hIgG4.SPK (V87E) (Table 4), therefore was selected as final lead for in vitro characterization. The sequences of the final PTM removed W3405 lead antibody, W3405-2.61.21-uAb-p1-hIgG4.SPK are shown in Table A, B and C.

TABLE 4
Human TIM-3 affinity of W3405-2.61.21-uAb-hIgG4.SPK
(V87E) and W3405-2.61.21-uAb-p1-hIgG4.SPK

| Target | Antibody | ka (1/Ms) | kd (1/s) | K_{D} (M) |
|---|---|---|---|---|
| hTIM-3.ECD.MBPHis | W3405-2.61.21-uAb-hIgG4.SPK (V87E) | 2.08E+05 | 3.18E−05 | 1.53E−10 |
| | W3405-2.61.21-uAb-p1-hIgG4.SPK | 2.32E+05 | 3.20E−05 | 1.38E−10 |

3.4 Human TIM-3 Affinity (SPR)

W3405-2.61.21-uAb-hIgG4.SPK (V87E) or W3405-2.61.21-uAb-p1-hIgG4.SPK binding affinity to human TIM-3 was detected by SPR assay using Biacore 8K. Each antibody was captured on an anti-human IgG Fc antibody immobilized CM5 sensor chip. Various concentrations of hTIM-3.ECD.MBPHis in running buffer (containing 0.9 mM CaCl₂)) were injected over the sensor chip at a flow rate of 30 μL/min for an association phase of 120 s, followed by 3600 s dissociation. The sensorgrams of blank surface and buffer channel were subtracted from the test sensorgrams. The experimental data were fitted by 1:1 model using Langmuir analysis.

### Example 4

### In Vitro Characterization

### 4.1 Human TIM-3 Binding (FACS)

Various concentrations of W3405 lead antibody, W3405-2.61.21-uAb-p1, positive and negative controls were added to hTIM-3-expressing transfectant cells, and then the binding of antibodies onto the surface of the cells was detected by PE-labeled goat anti-human IgG-Fc antibody. MFI of the cells was measured by a flow cytometer and analyzed by FlowJo.

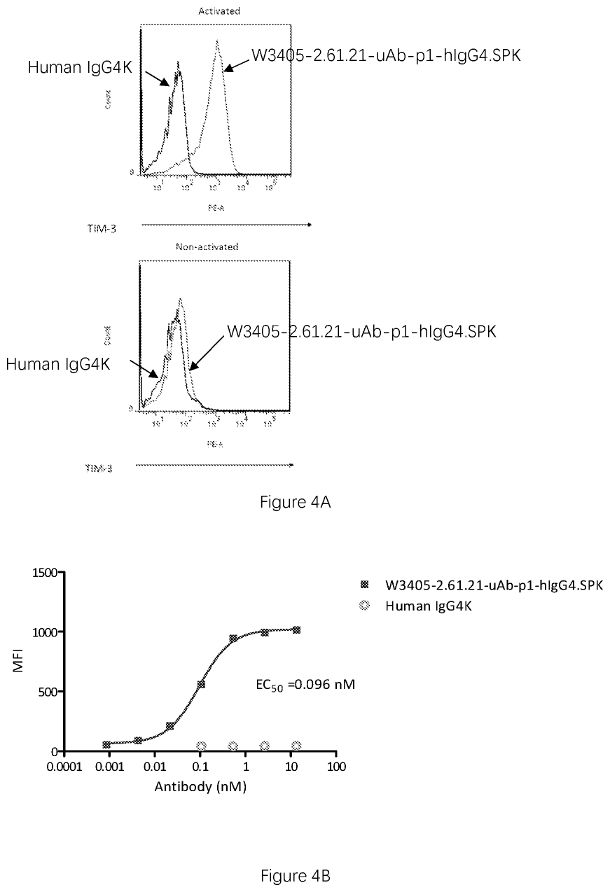

The binding of W3405 lead antibody, W3405-2.61.21-uAb-p1-hIgG4.SPK, on human TIM-3 transfected cells is shown in FIG. 3. The antibody strongly bound to cell surface human TIM-3 with an EC₅₀ of 0.13 nM.

### 4.2 Resting and Activated Human CD4⁺ T Cell Binding

It is known that TIM-3 expression can be induced on human CD4⁺ T cells post in vitro activation [14]. To determine whether W3405 lead antibody can bind to natural human TIM-3, freshly purified human CD4⁺ T cells were activated to induce TIM-3 expression.

Human peripheral blood mononuclear cells (PBMCs) were freshly isolated from healthy donors using Ficoll-Paque PLUS gradient centrifugation. Human CD4⁺ T cells were isolated using Human CD4⁺ T Cell Enrichment Kit according to the manufacturer's protocol. Purified human CD4⁺ T cells were stimulated with PHA or left unstimulated for three days. Various concentrations of the lead antibody, as well as negative control, were added to resting or activated human CD4⁺ T cells, and then the binding of antibodies onto the surface of the human CD4⁺ T cells was detected by PE-labeled goat anti-human IgG-Fc antibody. MFI of the cells was measured by a flow cytometer and analyzed by FlowJo.

As shown in FIG. 4, W3405 lead antibody, W3405-2.61.21-uAb-p1-hIgG4.SPK, bound to activated, but not resting CD4⁺ T cells. FIG. 4A shows the binding of the lead antibody on activated and non-activated CD4⁺ T cells. The binding curve of the lead antibody on activated CD4⁺ T cells is shown in FIG. 4B.

### 4.3 Paralog Binding (ELISA)

To test whether it specifically binds to human TIM-3, but not cross-reacts to the other TIM family members, the binding of W3405 lead antibody to human TIM-1 and TIM-4 was determined by ELISA. Lead antibody, positive and negative control antibodies were added to the plates that were pre-coated with either human TIM-1 or TIM-4. The binding of the antibodies to the plates was detected by corresponding HRP-conjugated secondary antibodies.

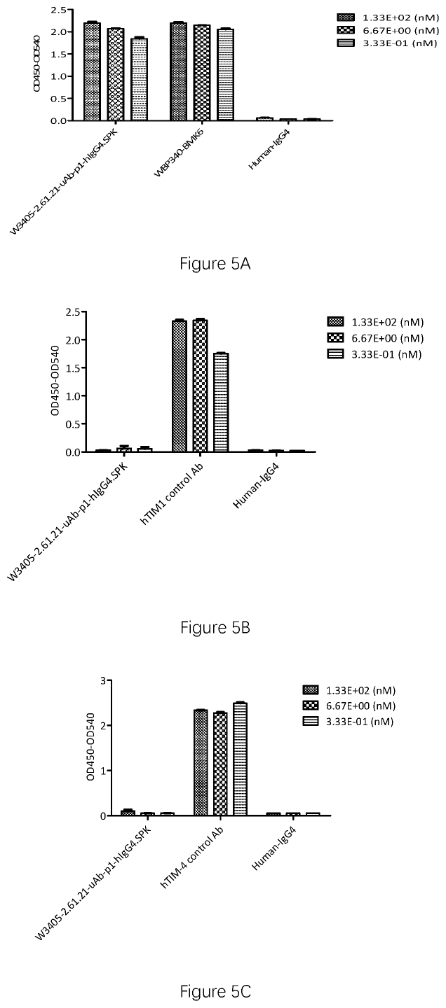

As shown in FIG. 5, W3405 lead antibody, W3405-2.61.21-uAb-p1-hIgG4.SPK, binds specifically to human TIM-3 (FIG. 5A), with no cross-reactive binding to human TIM-1 (FIG. 5B) or TIM-4 (FIG. 5C).

### 4.4 Cross-Species Binding (FACS)

The binding of the lead antibody to cynomolgus monkey TIM-3 was determined by FACS. Various concentrations of lead antibody, positive and negative controls were added to cynoTIM-3-expressing transfectant cells, and then the binding of antibodies onto the surface of the cells was detected by PE-labeled goat anti-human IgG-Fc antibody. MFI of the cells was measured by a flow cytometer and analyzed by FlowJo.

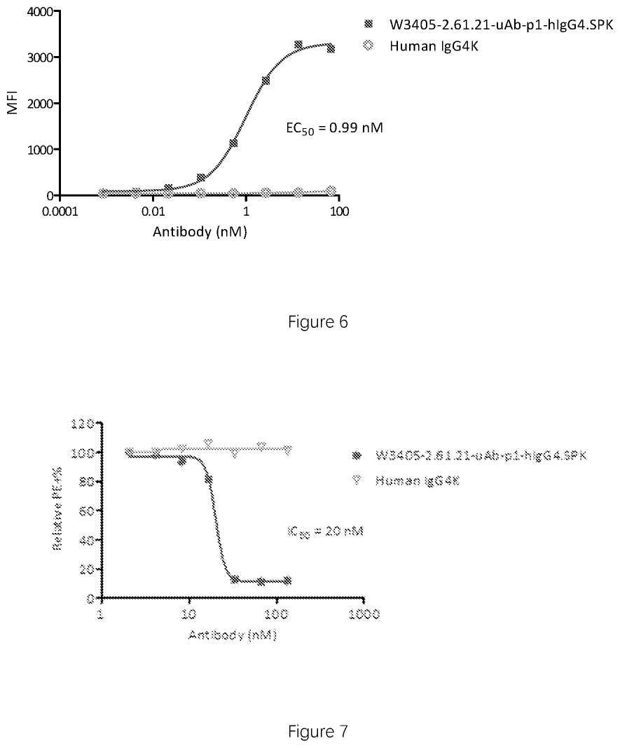

The binding result of W3405 lead antibody, W3405-2.61.21-uAb-p1-hIgG4.SPK, to cynomolgus monkey TIM-3 is shown in FIG. 6. The antibody showed strong binding to cynomolgus monkey TIM-3 with an EC₅₀ of 0.99 nM.

### 4.5 Cynomolgus Monkey TIM-3 Avidity (SPR)

W3405-2.61.21-uAb-p1-IgG4.SPK binding avidity to cynomolgus monkey TIM-3 was detected by SPR assay using Biacore 8K. The CM5 sensor chip (GE) was immobilized with cynoTIM-3.ECD.Fc. Testing antibody at different concentrations was injected over the sensor chip at a flow rate of 30 uL/min for an association phase of 200 s, followed by 2400 s dissociation. The sensorgrams of blank surface and buffer channel were subtracted from the test sensorgrams. The experimental data were fitted by 1:1 model using Langmuir analysis. The result is shown in Table 5.

TABLE 5
Cynomolgus monkey TIM-3 avidity of antibodies

| Target | Antibody | ka (1/Ms) | kd (1/s) | K_{D} (M) |
|---|---|---|---|---|
| CynoTIM-3.ECD.Fc | W3405-2.61.21-uAb-hIgG4.SPK (V87E) | 1.66E+06 | 7.49E−06 | 4.52E−12 |
| | W3405-2.61.21-uAb-p1-hIgG4.SPK | 2.89E+06 | 1.73E−05 | 6.00E−12 |

### 4.6 PtdSer (Phosphatidylserine) Competition Assay

It has been suggested by Sabatos-Peyton et al. that blockade of PtdSer is a shared property of anti-TIM-3 antibodies with demonstrated functional efficacy [15]. To determine whether W3405 lead antibody can block the binding between human TIM-3 and PtdSer, the Jurkat E6-1 cells were induced for apoptosis. The binding of human TIM-3 onto the surface of the apoptotic Jurkat cells was examined in the presence of various concentrations of W3405 lead antibody.

Jurkat E6-1 cells were treated with paclitaxel for 2 days to induce apoptosis. Various concentrations of lead antibody, positive and negative controls were pre-mixed with mFc-tagged human TIM-3 and then added to apoptotic Jurkat cells. The binding of human TIM-3 onto the surface of the apoptotic Jurkat cells was detected by PE-labeled anti-mouse IgG Fc antibody. MFI of the cells was measured by a flow cytometer and PE positive percent was analyzed by FlowJo.

As shown in FIG. 7, W3405 lead antibody, W3405-2.61.21-uAb-p1-hIgG4.SPK, demonstrates a dose-dependent blockade of PtdSer-TIM-3 interaction with an IC₅₀ of 20 nM.

### 4.7 Reporter Gene Assay

It has been implicated by Ferris et al. that TIM-3 may contribute to T cell exhaustion by enhancing TCR signaling, at least under acute conditions [16]. To test whether W3405 lead antibody can functionally counteract the role of TIM-3 in regulating T cell response, Jurkat E6-1 cells, which were stably integrated with IL-2 luciferase reporter gene, were transfected to express human TIM-3. The TIM-3⁺ Jurkat cells were activated by anti-CD28 antibody and anti-CD3 antibody in the presence of various concentrations of testing antibodies overnight at 37° C., 5% CO₂. After incubation, reconstituted luciferase substrate was added and the luciferase intensity was measured by a microplate spectrophotometer.

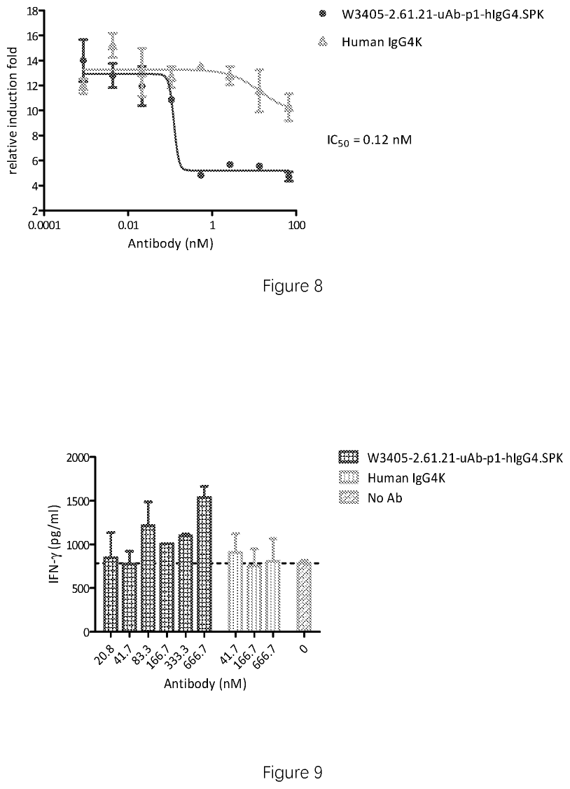

Consistent with Ferris' finding, the TIM-3-overexpressing Jurkat cells showed increased IL-2 reporter gene signal post anti-CD3/CD28 stimulation. As shown in FIG. 8, W3405 lead antibody, W3405-2.61.21-uAb-p1-hIgG4.SPK, can block the effect of TIM-3 on Jurkat cell IL-2 production in a dose dependent manner.

### 4.8 Allogeneic Mixed Lymphocyte Reaction (MLR)

PBMCs and human CD4⁺ T cells were isolated and purified as described above. Monocytes were isolated using CD14 MicroBeads according to the manufacturer's instructions. Cells were cultured in medium containing GM-CSF and IL-4 for 5 to 7 days to generate dendritic cells (DC). Purified CD4⁺ T cells were co-cultured with allogeneic mature DCs (mDCs) together with various concentrations of lead antibody in 96-well plates. On Day 5, the culture supernatants were harvested for IFNγ tests.

The results shown in FIG. 9 demonstrates that W3405 lead antibody, W3405-2.61.21-uAb-p1-hIgG4.SPK, can enhance the IFNγ production by human CD4⁺ T cells in a dose dependent manner.

### 4.9 T Cell Exhaustion Prevention Assay

As reported by Ozkazanc D. et al., co-culture with myeloid leukemia cells led to functional exhaustion of human CD4⁺ T cells [17]. To determine whether W3405 lead antibody can prevent THP-1 induced CD4⁺ T cell exhaustion, freshly isolated human CD4⁺ T cells were co-cultured with THP-1 cells in the presence of anti-CD3 antibody for 4-5 days to induce exhaustion. Various concentrations of lead antibody or isotype control were added to the culture to prevent the exhaustion of the T cells. On day 5, cells were harvested and stimulated with PMA/Ionomycin with Golgi-stop for 6 hours. The TL-2 production was determined by intracellular staining.

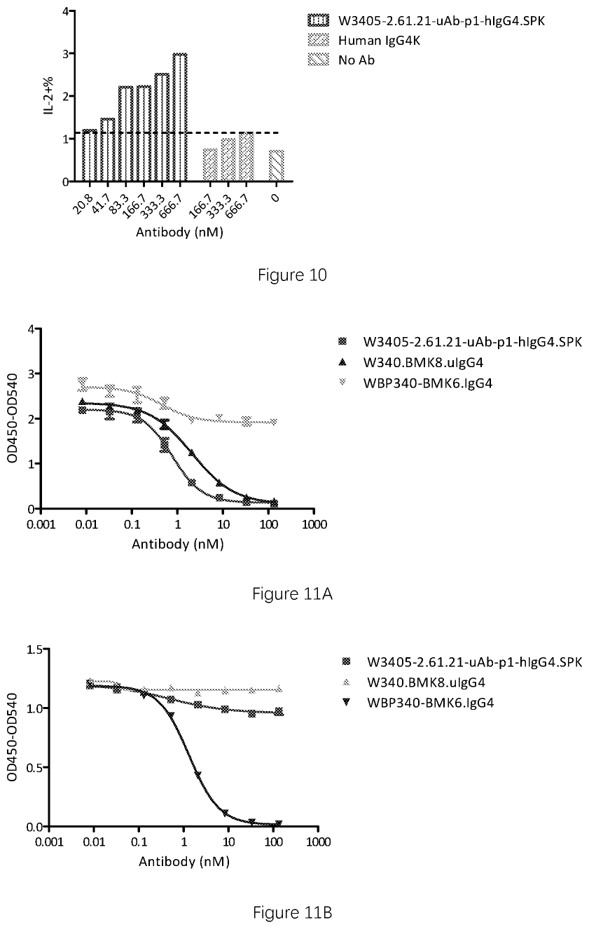

The results are shown in FIG. 10. In a dose-dependent manner, W3405 lead antibody, W3405-2.61.21-uAb-p1-hIgG4.SPK, can prevent the loss of IL-2 production by CD4⁺ T cells co-cultured with THP-1 cells.

### 4.10 Epitope Binning

Anti-human TIM-3 reference antibodies WBP340-BMK8 and WBP340-BMK6 were generated according to the sequences published in U.S. Pat. No. 9,605,070 B2 and US Patent Application No. US20160200815 A1, respectively. Various concentrations of testing antibodies were mixed with certain amount of biotinylated WBP340-BMK8 and W340-BMK6, respectively. The mixtures were then added to the plates pre-coated with human TIM-3 protein. The binding of BMK8 and BMK6 to the plates was detected by SA-HRP.

As shown in FIG. 11, W3405 lead antibody, W3405-2.61.21-uAb-p1-hIgG4.SPK, competes with WBP340-BMK8 (FIG. 11A), but not BMK6 (FIG. 11B), for binding to human TIM-3.

### 4.11 ADCC Assay

NK cells were isolated using Human CD56 MicroBeads according to the manufacturer's protocol. Human TIM-3 expressing CHO cells and various concentrations of testing antibodies were pre-incubated in 96-well plate for 30 minutes, and then NK cells were added at the effector/target ratio of 5:1. The plate was kept at 37° C. in a 5% CO₂ incubator for 4-6 hours. Target cell lysis was determined by LDH-based cytotoxicity detection kit. Herceptin induced ADCC effect on SKBR-3 cells was used as positive control.

### 4.12 CDC Assay

Human TIM-3 expressing CHO cells and various concentrations of testing antibodies were mixed in 96-well plates. Human complement was added at a final dilution of 1:50. The plates were kept at 37° C. in a 5% CO₂ incubator for 2-3 hours. Target cell lysis was determined by CellTiter-Glo. Rituxan®-induced Raji cell lysis was used as positive control.

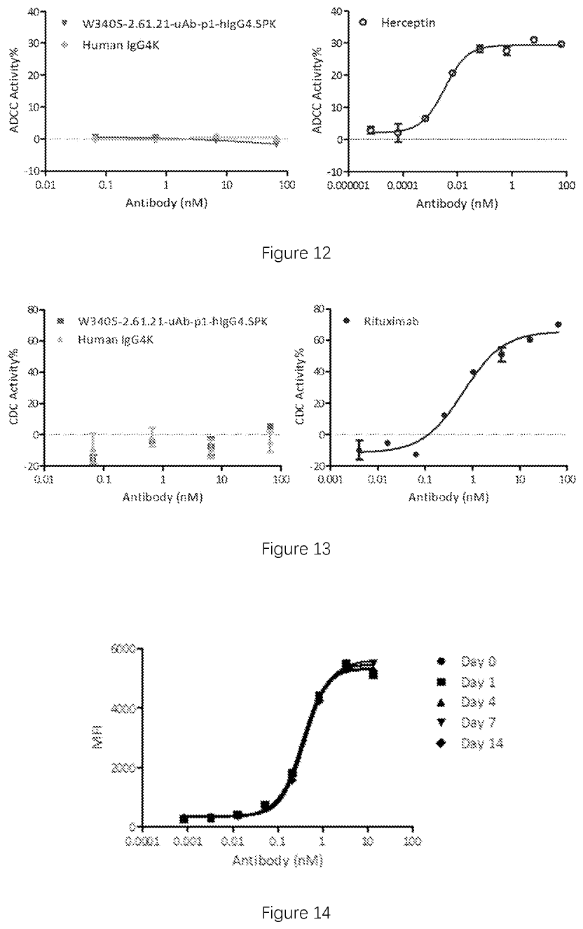

The results of ADCC (FIG. 12) and CDC (FIG. 13) suggest that W3405 lead antibody, W3405-2.61.21-uAb-p1, does not mediate ADCC or CDC activity on hTIM-3-expressing cells, which can avoid the potential damage to TIM-3 positive cells while it is used to treat patients.

### 4.13 Serum Stability

Testing antibody was 1:10 diluted in freshly collected human serum, aliquoted and cultured at 37° C. in a 5% CO₂ incubator. At indicated time point, an aliquot of the testing antibody was removed from culture, snap frozen, and then kept at −20° C., until ready for binding titration test by FACS as described above.

FIG. 14 suggests that W3405 lead antibody, W3405-2.61.21-uAb-p1-hIgG4.SPK, is stable in human serum at 37° C. for at least 14 days.

### Example 5

### In Vivo Characterization

### 5.1 Efficacy Study in NOG Mice HCC827 MiXeno™ Model

The therapeutic efficacy of W3405-2.61.21-uAb-p1-hIgG4.SPK was evaluated in HCC827 MiXeno™ model using NOG mice. On day 0, 5×10⁶ human non-small cell lung cancer HCC-827 cells were implanted subcutaneously into NOG mice (6-8 weeks old, female, Beijing Vital River). When tumors reached about 280 mm³, the animals were randomized and infused (i.v.) with 2.5×10⁶ activated human T cells. Post T cell infusion, mice were injected (i.p., weekly×4 weeks) with either W3405-2.61.21-uAb-p1-hIgG4.SPK or isotype control antibody (10 mg/kg). Tumor size was measured at least twice weekly. The tumor volume and TGI were calculated as follows. Tumor size was measured twice weekly in two dimensions using a caliper, and the volume was expressed in mm3 using the formula: V=0.5 a ×b² where a and b are the long and short diameters of the tumor, respectively. TGI was calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the isotype control group on the same day with Ti, and V0 is the average tumor volume of the isotype control group on the day of treatment start.

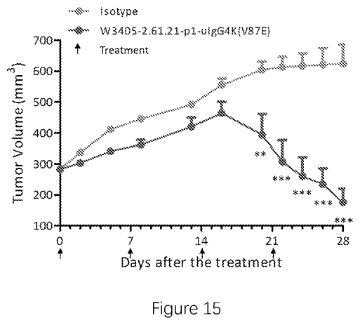

As shown in FIG. 15, from Day 0 to Day 16, W3405-2.61.21-uAb-p1-hIgG4.SPK treated animals showed a delay in tumor progression, as compared to isotype treated animals. Post the 3^{rd} dose of the treatment, animals received W3405-2.61.21-uAb-p1-hIgG4.SPK treatment started to show a significant and durable tumor regression. On day 28, i.e. 7 days post the last dose, the animals in the treatment group reached an average TGI of 131.4%, with 7/10 animals showed at least 40% tumor reduction from treatment initiation.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present invention is not limited to the particular embodiments that have been described in detail herein. Rather, reference should be made to the appended claims as indicative of the scope and content of the invention.

### REFERENCES

[1] Hafler D A and Kuchroo V. TIMs: central regulators of immune responses. J Exp Med. 2008; 205:2699-701.
[2] Jiang Y, Li Y, Zhu B. T-cell exhaustion in the tumor microenvironment. Cell Death Dis. 2015; 6:e1792.
[3] Wherry E J and Kurachi M. Molecular and cellular insights into T cell exhaustion. Nat Rev Immunol. 2015; 15:486-99.
[4] Tsai H F, Hsu P N. Cancer immunotherapy by targeting immune checkpoints: mechanism of T cell dysfunction in cancer immunity and new therapeutic targets. J Biomed Sci. 2017; 24:35.
[5] Cao E, et al. T cell immunoglobulin mucin-3 crystal structure reveals a galectin-9-independent ligand-binding surface. Immunity. 2007; 26:311-21.
[6] Huang Y H, et al. CEACAM1 regulates TIM-3-mediated tolerance and exhaustion. Nature. 2015; 517:386-90.
[7] DeKruyff R H, et al. T cell/transmembrane, Ig, and mucin-3 allelic variants differentially recognize phosphatidylserine and mediate phagocytosis of apoptotic cells. J Immunol. 2010; 184:1918-30.
[8] Chiba S, et al. Tumor-infiltrating DCs suppress nucleic acid-mediated innate immune responses through interactions between the receptor TIM-3 and the alarmin HMGBL. Nat Immunol. 2012; 13:832-42.
[9] Zhu C, et al. The Tim-3 ligand galectin-9 negatively regulates T helper type 1 immunity. Nat Immunol. 2005; 6:1245-52.
[10] Das M, Zhu C, and Kuchroo V K. Tim-3 and its role in regulating anti-tumor immunity. Immunol Rev. 2017; 276: 97-111.
[11] Kang C W, et al. Apoptosis of tumor infiltrating effector TIM-3+CD8⁺ T cells in colon cancer. Sci Rep. 2015; 5:15659.
[12] Fourcade J, et al. Upregulation of Tim-3 and PD-1 expression is associated with tumor antigen-specific CD8⁺ T cell dysfunction in melanoma patients. J Exp Med. 2010; 207:2175-86.
[13] Sakuishi K, et al. Targeting Tim-3 and PD-1 pathways to reverse T cell exhaustion and restore anti-tumor immunity. J Exp Med. 2010; 207:2187-94.
[14] Hastings W D, et al. TIM-3 is expressed on activated human CD4⁺ T cells and regulates Th1 and Th17 cytokines. Eur J Immunol. 2009; 39:2492-501.
[15] Sabatos-Peyton C A, et al. Blockade of Tim-3 binding to phosphatidylserine and CEACAM1 is a shared feature of anti-Tim-3 antibodies that have functional efficacy. Oncoimmunology. 2017; 7: e1385690.
[16] Ferris R L, Lu B, Kane L P. Too much of a good thing? Tim-3 and TCR signaling in T cell exhaustion. J Immunol. 2014; 193: 1525-30.
[17] Ozkazanc D, et al. Functional exhaustion of CD4⁺ T cells induced by co-stimulatory signals from myeloid leukaemia cells. Immunology. 2016; 149: 460-71.

1. An isolated antibody or an antigen-binding portion thereof, wherein the isolated antibody or the antigen-binding portion thereof comprises:
A) one or more heavy chain CDRs (HCDRs) selected from the group consisting of:
(i) a HCDR1 comprising SEQ ID NO: 1;
(ii) a HCDR2 comprising one of the amino acid sequences selected from the group consisting of SEQ ID NOs: 7 and 2; and
(iii) a HCDR3 comprising SEQ ID NO: 3;
B) one or more light chain CDRs (LCDRs) selected from the group consisting of:
(i) a LCDR1 comprising SEQ ID NO: 4;
(ii) a LCDR2 comprising SEQ ID NO: 5; and
(iii) a LCDR3 comprising SEQ ID NO: 6; or
C) one or more HCDRs of A) and one or more LCDRs of B).

2. The isolated antibody or the antigen-binding portion thereof of claim 1, wherein the isolated antibody or the antigen-binding portion thereof comprises:
A) one or more heavy chain CDRs (HCDRs) selected from the group consisting of:
(i) a HCDR1 as set forth in SEQ ID NO: 1;
(ii) a HCDR2 as set forth in one of the amino acid sequences selected from the group consisting of SEQ ID NOs: 7 and 2; and
(iii) a HCDR3 as set forth in SEQ ID NO: 3;
B) one or more light chain CDRs (LCDRs) selected from the group consisting of:
(i) a LCDR1 as set forth in SEQ ID NO: 4;
(ii) a LCDR2 as set forth in SEQ ID NO: 5; and
(iii) a LCDR3 as set forth in SEQ ID NO: 6; or
C) one or more HCDRs of A) and one or more LCDRs of B).

3. The isolated antibody or the antigen-binding portion thereof of claim 1, wherein the isolated antibody or the antigen-binding portion thereof comprises a heavy chain variable region (VH) and a light chain variable region (VL), and wherein
(a) the VH comprises:
(i) a HCDR1 as set forth in SEQ ID NO: 1;
(ii) a HCDR2 as set forth in SEQ ID NO: 7 or 2; and
(iii) a HCDR3 as set forth in SEQ ID NO: 3; and
(b) the VL comprises:
(i) a LCDR1 as set forth in SEQ ID NO: 4;
(ii) a LCDR2 as set forth in SEQ ID NO: 5; and
(iii) a LCDR3 as set forth in SEQ ID NO: 6.

4. (canceled)

5. The isolated antibody or the antigen-binding portion thereof of claim 1, wherein the isolated antibody or the antigen-binding portion thereof comprises:
(A) a heavy chain variable region (VH):
(i) comprising the amino acid sequence selected from the group consisting of SEQ ID NOs: 14 and 8;
(ii) comprising an amino acid sequence at least 85%, 90%, or 95% identical to the amino acid sequence selected from the group consisting of SEQ ID NOs: 14 and 8; or
(iii) comprising an amino acid sequence with addition, deletion and/or substitution of one or more amino acids compared with the amino acid sequence selected from the group consisting of SEQ ID NOs: 14 and 8; and/or
(B) a light chain variable region (VL):
(i) comprising the amino acid sequence selected from the group consisting of SEQ ID NOs: 12 and 10;
(ii) comprising an amino acid sequence at least 85%, at least 90%, or at least 95% identical to the amino acid sequence selected from the group consisting of SEQ ID NOs: 12 and 10; or
(iii) comprising an amino acid sequence with addition, deletion and/or substitution of one or more amino acids compared with the amino acid sequence selected from the group consisting of SEQ ID NOs: 12 and 10.

6. The isolated antibody or the antigen-binding portion thereof of claim 5, wherein the isolated antibody or the antigen-binding portion thereof comprises:
(a) a heavy chain variable region comprising or consisting of the amino acid sequence of SEQ ID NO: 14_and a light chain variable region comprising or consisting of the amino acid sequence of SEQ ID NO: 12; or
(b) a heavy chain variable region comprising or consisting of the amino acid sequence of SEQ ID NO: 8 and a light chain variable region comprising or consisting of the amino acid sequence of SEQ ID NO: 10; or
(c) a heavy chain variable region comprising or consisting of the amino acid sequence of SEQ ID NO: 8 and a light chain variable region comprising or consisting of the amino acid sequence of SEQ ID NO: 12.

7.-10. (canceled)

11. The isolated antibody or the antigen-binding portion thereof of claim 1, having one or more of the following properties:
(a) specifically binding to both human TIM-3 protein and cynomolgus monkey TIM-3 protein;
(b) blocking the binding of TIM3 to PtdSer;
(c) enhancing TCR signaling; and
(d) inducing production of a cytokine in human CD4+T cells.

12. The isolated antibody or the antigen-binding portion thereof of claim 1, wherein the antibody is a chimeric antibody, a humanized antibody or a fully human antibody.

13. (canceled)

14. The isolated antibody or the antigen-binding portion thereof of claim 1, wherein the antibody comprises constant region of a human IgG.

15. The isolated antibody or the antigen-binding portion thereof of claim 1, wherein the antibody comprises constant region of a human IgG4 isotype comprising a S228P mutation.

16. An isolated nucleic acid molecule, comprising a nucleic acid sequence encoding the heavy chain variable region and/or the light chain variable region of the isolated antibody as defined in claim 1.

17.-18. (canceled)

19. A vector comprising the nucleic acid molecule of any claim 16.

20. A host cell comprising the vector of claim 19.

21. A pharmaceutical composition comprising the antibody or the antigen-binding portion thereof as defined in claim 1 and a pharmaceutically acceptable carrier.

22. A method for producing the antibody or the antigen-binding portion thereof as defined in claim 1, comprising the steps of:
expressing the antibody or antigen-binding portion thereof in a host cell comprising a nucleic acid molecule(s) encoding the antibody or antigen-binding portion thereof; and
isolating the antibody or antigen-binding portion thereof from the host cell.

23. A method of modulating an immune response in a subject, comprising administering to the subject the antibody or the antigen-binding portion thereof as defined in claim 1 such that an immune response is modulated in the subject.

24. (canceled)

25. A method for treating abnormal cell growth, inhibiting growth of tumor cells or reducing tumor cell metastasis in a subject, comprising administering an effective amount of the antibody or the antigen-binding portion thereof as defined in claim 1 to the subject.

26. (canceled)

27. A method for treating or preventing diseases comprising cancers, immune disorders, inflammatory diseases or infectious diseases in a subject, comprising administering an effective amount of the antibody or the antigen-binding portion thereof as defined in claim 1 to the subject.

28. The method of claim 27, wherein the cancer is colon cancer or lung cancer.

29. The method of claim 28, wherein the lung cancer is non-small cell lung cancer.

30.-38. (canceled)

39. A kit for treating or diagnosing proliferative disorders, autoimmune diseases, immune disorders or infectious diseases, comprising a container comprising at least one antibody or the antigen-binding portion thereof as defined in claim 1.


SEQUENCE LISTING (15 sequences)

SEQ ID NO: 1 - protein, 10 aa, Artificial Sequence
  VH CDR1 amino acid sequence
GFTFSNYAMS

SEQ ID NO: 2 - protein, 17 aa, Artificial Sequence
  VH CDR2 amino acid sequence
SISDNGGTAYHADSVQG

SEQ ID NO: 3 - protein, 8 aa, Artificial Sequence
  VH CDR3 amino acid sequence
DFGDSPGY

SEQ ID NO: 4 - protein, 17 aa, Artificial Sequence
  VL CDR1 amino acid sequence
KSSQSVLYSFKNKNYLA

SEQ ID NO: 5 - protein, 7 aa, Artificial Sequence
  VL CDR2 amino acid sequence
WASTRES

SEQ ID NO: 6 - protein, 9 aa, Artificial Sequence
  VL CDR3 amino acid sequence
QQYYSSPLT

SEQ ID NO: 7 - protein, 17 aa, Artificial Sequence
  VH CDR2 amino acid sequence
SISDQGGTAYHADSVQG

SEQ ID NO: 8 - protein, 117 aa, Artificial Sequence
  VH amino acid sequence
EVQLLEAGGGPVQPGGSLRLSCAAAGFTFSNYAMSWVRQAPGKGLEWVSSISDNGGTAYHADSVQGRFTISRDNSKSTLYLQMNSLRAEDTAVYYCAKDFGDSPGYWGQGTLVTVSS

SEQ ID NO: 9 - DNA, 351 bp, Artificial Sequence
  VH nucleic acid sequence
gaggtgcagttgttggaggctgggggaggcccggtacagcctggggggtccctgagactctcctgtgcagccgctggattcacctttagcaactatgccatgagctgggtccggcaggctccagggaaggggctggaatgggtctcaagtattagtgacaatggtgggaccgcataccacgcagactccgtgcagggccgattcaccatctccagagacaattccaagagcacgctgtatctacaaatgaacagcctgagagccgaggacacggccgtatattactgtgcgaaagacttcggtgactccccgggctactggggccagggaaccctggtcaccgtctcctca

SEQ ID NO: 10 - protein, 113 aa, Artificial Sequence
  VL amino acid sequence
DIVMTQSPDSLAVSLGERATINCKSSQSVLYSFKNKNYLAWYQQKPGQPPKLLIYWASTRESGVPDRFSGSGSGTDFTLTISSLQAVDVAVYYCQQYYSSPLTFGGGTKVEIK

SEQ ID NO: 11 - DNA, 339 bp, Artificial Sequence
  VL nucleic acid sequence
gacatcgtgatgacccagtctccagactccctggctgtgtctctgggcgagagggccaccatcaactgcaagtccagccagagtgttttatacagcttcaagaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgtagatgtggcagtttattactgtcagcaatattatagttctccgctcactttcggcggagggaccaaggtggagatcaaa

SEQ ID NO: 12 - protein, 113 aa, Artificial Sequence
  VL amino acid sequence
DIVMTQSPDSLAVSLGERATINCKSSQSVLYSFKNKNYLAWYQQKPGQPPKLLIYWASTRESGVPDRFSGSGSGTDFTLTISSLQAEDVAVYYCQQYYSSPLTFGGGTKVEIK

SEQ ID NO: 13 - DNA, 339 bp, Artificial Sequence
  VL nucleic acid sequence
gacatcgtgatgacccagtctccagactccctggctgtgtctctgggcgagagggccaccatcaactgcaagtccagccagagtgttttatacagcttcaagaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagttctccgctcactttcggcggagggaccaaggtggagatcaaa

SEQ ID NO: 14 - protein, 117 aa, Artificial Sequence
  VH amino acid sequence
EVQLLEAGGGPVQPGGSLRLSCAAAGFTFSNYAMSWVRQAPGKGLEWVSSISDQGGTAYHADSVQGRFTISRDNSKSTLYLQMNSLRAEDTAVYYCAKDFGDSPGYWGQGTLVTVSS

SEQ ID NO: 15 - DNA, 351 bp, Artificial Sequence
  VH nucleic acid sequence
gaggtgcagttgttggaggctgggggaggcccggtacagcctggggggtccctgagactctcctgtgcagccgctggattcacctttagcaactatgccatgagctgggtccggcaggctccagggaaggggctggaatgggtctcaagtattagtgaccagggtgggaccgcataccacgcagactccgtgcagggccgattcaccatctccagagacaattccaagagcacgctgtatctacaaatgaacagcctgagagccgaggacacggccgtatattactgtgcgaaagacttcggtgactccccgggctactggggccagggaaccctggtcaccgtctcctca